(19) United States
(12) Patent Application Publication
(10) Pub. No.: US 2024/0149051 A1
(43) Pub. Date: May 9, 2024

(54) SYSTEMS AND METHODS FOR AUXILIARY DISPLAY OF HEMODYNAMIC SIGNALS OR INDICATORS

(71) Applicant: Abiomed, Inc., Danvers, MA (US)

(72) Inventors: Erik Ian Kroeker, Danvers, MA (US); Muhammad Sami, Danvers, MA (US); Christian Moyer, Danvers, MA (US); Ahmad El Katerji, Danvers, MA (US)

(73) Assignee: Abiomed, Inc., Danvers, MA (US)

(21) Appl. No.: 18/503,478

(22) Filed: Nov. 7, 2023

Related U.S. Application Data
(60) Provisional application No. 63/423,195, filed on Nov. 7, 2022.
(60) Provisional application No. 63/449,818, filed on Mar. 3, 2023.
(60) Provisional application No. 63/496,357, filed on Apr. 14, 2023.
(60) Provisional application No. 63/515,810, filed on Jul. 26, 2023.

(51) Int. Cl. A61M 60/515; A61M 60/585; G16H 50/30; G16H 50/70

(52) U.S. Cl. CPC A61M 60/515; A61M 60/585; G16H 50/30; G16H 50/70; A61M 2205/18; A61M 2205/35; A61M 2205/502; A61M 2230/005

(57) ABSTRACT

Methods and apparatus for an auxiliary monitor for a patient monitoring system including a first patient monitoring device and a second patient monitoring device are described. The auxiliary monitor comprises a communication interface configured to receive first data from the first patient monitoring device and second data from the second patient monitoring device, and at least one computer processor. The at least one computer processor is programmed to display in a first portion of a user interface, an indication of the first data, and display in a second portion of the user interface, an indication of the second data.

### CROSS-REFERENCE TO RELATED APPLICATIONS

This application claims the benefit under 35 U.S.C. § 119(e) to U.S. Provisional Patent Application No. 63/423,195, filed Nov. 7, 2022, and titled, “SYSTEMS AND METHODS FOR AUXILIARY DISPLAY OF HEMODYNAMIC SIGNALS OR INDICATORS,” and claims priority to U.S. Provisional Patent Application No. 63/449,818, filed Mar. 3, 2023, and titled “SYSTEMS AND METHODS FOR AUXILIARY DISPLAY OF HEMODYNAMIC SIGNALS OR INDICATORS,” and claims priority to U.S. Provisional Patent Application No. 63/496,357, filed Apr. 14, 2023, titled “SYSTEMS AND METHODS FOR AUXILIARY DISPLAY OF HEMODYNAMIC SIGNALS OR INDICATORS,” and claims priority to U.S. Provisional Patent Application No. 63/515,810, filed Jul. 26, 2023, titled “SYSTEMS AND METHODS FOR AUXILIARY DISPLAY OF HEMODYNAMIC SIGNALS OR INDICATORS,” the entire contents of each of which is incorporated by reference herein.

### FIELD OF THE INVENTION

This disclosure relates to techniques for display of hemodynamic signals or indicators on an auxiliary device.

### BACKGROUND

Fluid pumps, such as blood pumps, are used in the medical field in a wide range of applications and purposes. An intravascular blood pump is a pump that can be advanced through a patient's vasculature, i.e., veins and/or arteries, to a position in the patient's heart or elsewhere within the patient's circulatory system. For example, an intravascular blood pump may be inserted via a catheter and positioned to span a heart valve. The intravascular blood pump is typically disposed at the end of the catheter. Once in position, the pump may be used to assist the heart and pump blood through the circulatory system and, therefore, temporarily reduce workload on the patient's heart, such as to enable the heart to recover after a heart attack. An exemplary intravascular blood pump is available from ABIOMED, Inc., Danvers, MA under the tradename Impella® heart pump.

Such pumps can be positioned, for example, in a cardiac chamber, such as the left ventricle, to assist the heart. In this case, the blood pump may be inserted via a femoral artery by means of a hollow catheter and introduced up to and into the left ventricle of a patient's heart. From this position, the blood pump inlet draws in blood and the blood pump outlet expels the blood into the aorta. In this manner, the heart's function may be replaced or at least assisted by operation of the pump.

An intravascular blood pump is typically connected to a respective external heart pump controller that controls the heart pump, such as motor speed, and collects and displays operational data about the blood pump, such as heart signal level, battery temperature, blood flow rate and plumbing integrity. An exemplary heart pump controller is available from ABIOMED, Inc. under the trade name Automated Impella Controller®. The controller may raise alarms when operational data values fall outside predetermined values or ranges, for example if a leak, suction, and/or pump malfunction is detected. The controller may include a video display screen upon which is displayed a graphical user interface configured to display the operational data and/or alarms.

### SUMMARY

Described herein are systems and methods for an auxiliary monitor for a medical device support system, such as a cardiac support system. The auxiliary monitor may be configured to receive data from one or more controllers (e.g., an Automated Impella Controller®) of a heart pump system or other medical device (e.g., a patient monitor, a respiratory monitor, a body temperature monitor, an extracorporeal membrane oxygenation (ECMO) machine, etc.). As will be appreciated, the auxiliary monitor may be configured to be directly connected to the one or more medical devices and/or from one or more remote web-based services (e.g., a cloud-based systems) in some embodiments such that the auxiliary monitor may receive data directly form the one or more medical devices and/or the web-based service(s).

In one aspect, an auxiliary monitor for a patient monitoring system including a first patient monitoring device and a second patient monitoring device is provided. The auxiliary monitor comprises a communication interface configured to receive first data from the first patient monitoring device and second data from the second patient monitoring device and at least one computer processor. The at least one computer processor is programmed to display in a first portion of a user interface, an indication of the first data and display in a second portion of the user interface, an indication of the second data.

In another aspect, the at least one computer processor is further programmed to process the first data and the second data to generate third data, and display in a third portion of the user interface, an indication of the third data. In another aspect, the third data includes a likelihood of organ failure for a patient being monitored by the patient monitoring system. In another aspect, the third data includes a cumulative metric.

In another aspect, the at least one computer processor is further programmed to display, in a third portion of the user interface, an alert, the alert determined based on the first data and/or the second data. In another aspect, the alert is determined based on the first data and the second data. In another aspect, the alert includes a recommendation to change an operation of first patient monitoring device and/or the second patient monitoring device.

In another aspect, the first patient monitoring device comprises a first controller for a first cardiac support device including a first heart pump, and the first data includes information describing operation of the first heart pump. In another aspect, the information describing operation of the first heart pump includes one or more of a pump speed, a flow rate of blood through the pump, or a pressure measurement. In another aspect, the second patient monitoring device comprises a patient monitor, and the second data includes vital signs measured by the patient monitor. In another aspect, the second patient monitoring device comprises a second controller for a second cardiac support device including a second heart pump, and the second data includes information describing operation of the second heart pump. In another aspect, the at least one computer processor is further programmed to process the first data and the second data to generate third data, and display in a third portion of the user interface, an indication of the third data. In another aspect, the third data includes a likelihood of heart failure for a patient being monitored by the patient monitoring system.

In another aspect, the communication interface is further configured to receive third data from a medical information source, and the third data including electronic health information for a patient being monitored by the patient monitoring system. In another aspect, the first patient monitoring device is associated with a first patient, and the second patient monitoring device is associated with a second patient.

In another aspect, the at least one processor is further programmed to issue a request via the communication interface to the first patient monitoring device and/or the second patient monitoring device, the request including a request for the first data and/or the second data, wherein the communication interface is configured to receive the first data and/or the second data in response to the request. In another aspect, the at least one processor is further programmed to issue a request via the communication interface to at least one network-connected processor, the request including a request to process the first data and/or the second data to generate processed data, the communication interface is configured to receive the processed data in response to the request, and the at least one processor is further programmed to display in a third portion of the user interface, an indication of the processed data. In another aspect, the communication interface is configured to be connected to a connectivity gateway, the connectivity gateway communicatively coupling the communication interface to the first patient monitoring device and the second patient monitoring device.

In another aspect, the auxiliary monitor further comprises at least one storage device configured to store one or more of the first data, the second data, or data derived from the first data and/or the second data. In another aspect, the data derived from the first data and/or the second data includes historical data associated with the first patient monitoring device and/or the second patent monitoring device.

In one aspect, a patient monitoring system is provided. The patient monitoring system comprises a first patient monitoring device, a second patient monitoring device, an auxiliary monitor comprising at least one computer processor and a display, and a connectivity gateway configured to communicatively couple the first patient monitoring device and the second monitoring device to the auxiliary monitor.

In another aspect, the first patient monitoring device comprises a first controller for a first cardiac support device. In another aspect, the second patient monitoring device comprises a second controller for a second cardiac support device. In another aspect, the patient monitoring system further comprises a third patient monitoring device communicatively coupled to the auxiliary monitor via the connectivity gateway, wherein the third patient monitoring device comprises a patient monitor configured to monitor vital signs of a patient. In another aspect, the first patient monitoring device comprises a patient monitor configured to monitor vital signs of a patient.

In another aspect, the at least one computer processor of the auxiliary monitor is programmed to display in a first portion of a user interface presented on the display, an indication of first data received from the first patient monitoring device via the connectivity gateway, and display in a second portion of the user interface, an indication of second data received from the second patient monitoring device via the connectivity gateway. In another aspect, the at least one computer processor of the auxiliary monitor is further programmed to process the first data and the second data to generate third data, and display in a third portion of the user interface, an indication of the third data. In another aspect, the patient monitoring system further comprises a medical information management system communicatively coupled to the auxiliary monitor via the connectivity gateway, the medical information management system including third data including electronic health information for a patient being monitored by the patient monitoring system.

In one aspect, a method of monitoring a first cardiac support device and a second cardiac device using an auxiliary monitor is provided. The method comprises receiving, from the first cardiac support device, first data associated with operation of the first cardiac support device, receiving, from the second cardiac support device, second data associated with operation of the second cardiac support device, displaying, on a first portion of a user interface of the auxiliary monitor, an indication of the first data, and displaying, on a second portion of the user interface, an indication of the second data.

In another aspect, the method further comprises processing the first data and the second data to generate third data, and displaying in a third portion of the user interface, an indication of the third data. In another aspect, processing the first data and the second data to generate third data comprises determining a likelihood of heart failure for a patient having the first cardiac device and the second cardiac device. In another aspect, the method further comprises determining, based on the first data and/or the second data, an alert, and displaying, in a third portion of the user interface, the alert. In another aspect, determining, based on the first data and/or the second data, an alert comprises determining the alert based on the first data and the second data.

In another aspect, the first data includes one or more of a pump speed, a flow rate of blood through the pump, or a pressure measurement. In another aspect, the method further comprises receiving, from a patient monitor, vital signs data, and displaying, in a third portion of the user interface, an indication of the vital signs data. In another aspect, the method further comprises receiving, from a medical information source, third data including electronic health information for a patient having the first cardiac support device and the second cardiac support device. In another aspect, the first cardiac support device is associated with a first patient, and the second cardiac support device is associated with a second patient.

In another aspect, the method further comprises issuing, to the first cardiac support device and/or the second cardiac support device, a request for the first data and/or the second data, and receiving the first data and/or the second data in response to issuing the request. In another aspect, the method further comprises issuing, to at least one network-connected processor, a request to process the first data and/or the second data to generate processed data, and receiving the processed data in response to issuing the request, displaying, in a third portion of the user interface, an indication of the processed data. In another aspect, receiving the first data from the first cardiac support device and receiving the second data from the second assist cardiac device comprises receiving the first data and the second data via a connectivity gateway communicatively coupling the auxiliary monitor to each of the first cardiac support device and the second cardiac support device.

In another aspect, the method further comprises storing, on at least one storage device one or more of the first data, the second data, or data derived from the first data and/or the second data. In another aspect, the method further comprises deriving from the first data and/or the second data, historical data associated with the first cardiac support device and/or the second cardiac support device, and storing the historical data on the at least one storage device. In another aspect, the method further comprises receiving, via the user interface of the auxiliary monitor, a request to reconfigure display of information on the user interface, and reconfiguring the display of information on the user interface of the auxiliary monitor in response to receiving the request. In another aspect, the request to reconfigure display of information on the user interface is a request to add an available patient monitoring device or a request to remove a connected patient monitoring device.

In one aspect, an auxiliary monitor for a patient monitoring system is provided. The auxiliary monitor comprises a communication interface configured to receive time-series data from at least one patient monitoring device, and at least one computer processor. The at least one computer processor is programmed to display in a first portion of a user interface, a timeline of events associated with the time-series data, and display in a second portion of the user interface, a plurality of widgets, each of the plurality of widgets being configured to display physiological data associated with the time-series data, information associated with operation of the at least one patient monitoring device, or information associated with operation of the auxiliary monitor.

### BRIEF DESCRIPTION OF DRAWINGS

FIG. 1 shows an illustrative cardiac support device that may be used to generate monitored data for use with some embodiments.

FIG. 2 shows an illustrative patient monitoring system that includes multiple patient monitoring devices and an auxiliary monitor, in accordance with some embodiments.

FIGS. 3A-3J illustrate portions of a user interface of an auxiliary monitor configured to display information from one or more connected patient monitoring devices, in accordance with some embodiments.

FIG. 4 is a flowchart of a process for displaying information from multiple patient monitoring devices on a user interface of an auxiliary monitor, in accordance with some embodiments.

FIG. 5 is a flowchart of a process for processing data from multiple patient monitoring devices to generate new data that may be displayed on a user interface of an auxiliary monitor, in accordance with some embodiments.

FIGS. 6A-6T illustrate portions of a user interface of an auxiliary monitor configured to display information from one or more connected patient monitoring devices, in accordance with some embodiments.

FIG. 7 shows a patient monitoring system that includes multiple patient monitoring devices, an auxiliary monitor, and an additional information source, in accordance with some embodiments.

FIG. 8 illustrates portions of a user interface of an auxiliary monitor, in accordance with one embodiment.

### DETAILED DESCRIPTION

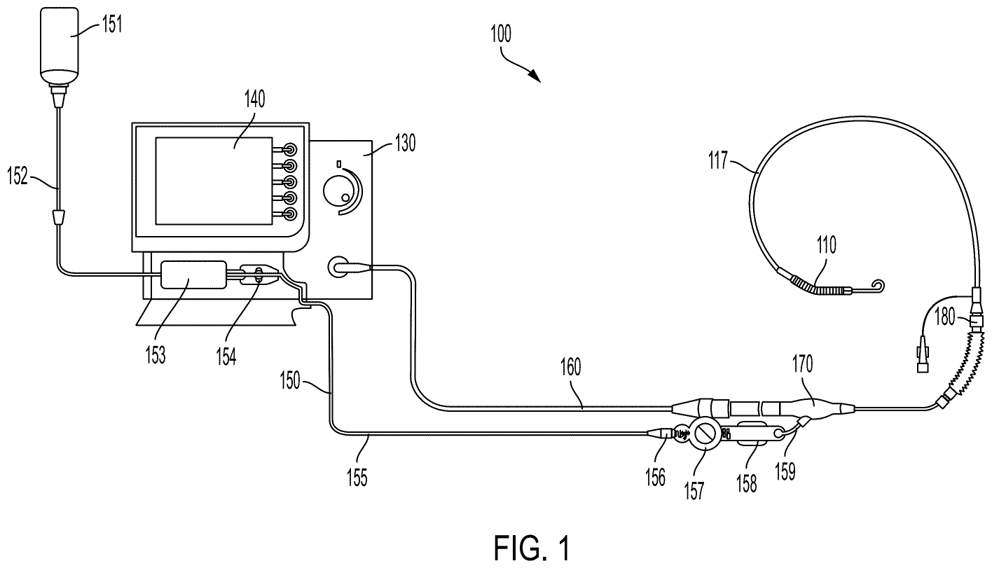

A circulatory support device (also referred to herein as a “heart pump” or simply a “pump”) may include a percutaneous, catheter-based device that provides hemodynamic support to the heart of a patient. As shown in FIG. 1, heart pump 110 may form part of a cardiac support system 100. Cardiac support system 100 also may include a controller 130 (e.g., an Automated Impella Controller®, referred to herein as an “AIC,” from ABIOMED, Inc, Danvers, Mass.), a display 140, a purge subsystem 150, a connector cable 160, a plug 170, and a repositioning unit 180. As shown, controller 130 may include display 140. Controller 130 may be configured to monitor and control operation of heart pump 110. During operation, purge subsystem 150 may be configured to deliver a purge fluid to heart pump 110 through catheter tube 117 to prevent blood from entering the motor (not shown) of the heart pump. In some implementations, the purge fluid is a dextrose solution (e.g., 5% dextrose in water with 25 or 50 IU/mL of heparin, although the solution need not include heparin in all embodiments). Connector cable 160 may provide an electrical connection between heart pump 110 and controller 130. Plug 170 may connect catheter tube 117, purge subsystem 150, and connector cable 160. In some implementations, plug 170 includes a storage device (e.g., a memory) configured to store, for example, operating parameters to facilitate transfer of the patient to another controller if needed. Repositioning unit 180 may be used to reposition heart pump 110 in the patient's heart.

As shown in FIG. 1, in some embodiments, the cardiac support system 100 may include a purge subsystem 150 having a container 151, a supply line 152, a purge cassette 153, a purge disc 154, purge tubing 155, a check valve 156, a pressure reservoir 157, an infusion filter 158, and a sidearm 159. Container 151 may, for example, be a bag or a bottle. As will be appreciated, in other embodiments the cardiac support system 100 may not include a purge subsystem. In some embodiments, a purge fluid may be stored in container 151. Supply line 152 may provide a fluidic connection between container 151 and purge cassette 153. Purge cassette 153 may control how the purge fluid in container 151 is delivered to heart pump 110. For example, purge cassette 153 may include one or more valves for controlling a pressure and/or flow rate of the purge fluid. Purge disc 154 may include one or more pressure and/or flow sensors for measuring a pressure and/or flow rate of the purge fluid. As shown, controller 130 may include purge cassette 153 and purge disc 154. Purge tubing 155 may provide a fluidic connection between purge disc 154 and check valve 156. Pressure reservoir 157 may provide additional filling volume during a purge fluid change. In some implementations, pressure reservoir 157 includes a flexible rubber diaphragm that provides the additional filling volume by means of an expansion chamber. Infusion filter 158 may help prevent bacterial contamination and air from entering catheter tube 117. Sidearm 159 may provide a fluidic connection between infusion filter 158 and plug 170. Although shown as having separate purge tubing and connector cable, it will be appreciated that in some embodiments, the cardiac support system 100 may include a single connector with both fluidic and electric lines connectable to the controller 130.

During operation, controller 130 may be configured to receive measurements from one or more pressure sensors (not shown) included as a portion of heart pump 110 and purge disc 154. Controller 130 may also be configured to control operation of the motor (not shown) of the heart pump 110 and purge cassette 153. As noted herein, controller 130 may be configured to control and measure a pressure and/or flow rate of a purge fluid via purge cassette 153 and purge disc 154. During operation, after exiting purge subsystem 150 through sidearm 159, the purge fluid may be channeled through purge lumens (not shown) within catheter tube 117 and plug 170. Sensor cables (not shown) within catheter tube 117, connector cable 160, and plug 170 may provide an electrical connection between components of the heart pump 110 (e.g., one or more pressure sensors) and controller 130. Motor cables (not shown) within catheter tube 117, connector cable 160, and plug 170 may provide an electrical connection between the motor of the heart pump 110 and controller 130. During operation, controller 130 may be configured to receive measurements from one or more pressure sensors of the heart pump 110 through the sensor cables (e.g., optical fibers) and to control the electrical power delivered to the motor of the heart pump 110 through the motor cables. By controlling the power delivered to the motor of the heart pump 110, controller 130 may be operable to control the speed of the motor.

Various modifications can be made to cardiac support system 100 and one or more of its components. For instance, one or more additional sensors may be added to heart pump 100. In another example, a signal generator may be added to heart pump 100 to generate a signal indicative of the rotational speed of the motor of the heart pump 110. As another example, one or more components of cardiac support system 100 may be separated. For instance, display 140 may be incorporated into another device in communication with controller 130 (e.g., wirelessly or through one or more electrical cables).

A heart pump (e.g., heart pump 110) may include a pressure sensor (e.g., an optical pressure sensor) configured to detect a pressure within the aorta of a patient's heart when the heart pump is properly positioned. The pressure signal sensed by the pressure sensor may be used, at least in part, to determine correct positioning of the heart pump within the patient's heart and/or to determine a blood flow rate through the heart pump when in operation. For instance, the pressure signal may be used in combination with a motor current signal received from a motor current sensor (not shown) and a set of stored values to determine a flow rate of blood through the heart pump. The differential pressure across the aortic valve may also indirectly be determined based on the pressure signal measuring the pressure in the aorta and the set of stored values.

Conventional medical devices include a controller and a display, which may be used to monitor one or more aspects of the operation of the medical device and/or the patient's physiology. The inventors have recognized and appreciated that when a patient is simultaneously using multiple medical devices (e.g., multiple patient monitoring devices), a healthcare worker (e.g., a physician) must monitor multiple controllers and displays to determine how the corresponding medical device is operating. Some embodiments of the technology described herein relate to an auxiliary monitor configured to receive data from multiple medical devices and display the data in a manner that provides a more intuitive and complete view of a patient's physiological condition when using the medical devices. For example, a physician may be able to monitor operation of multiple devices on a single display rather than having to monitor multiple displays connected to individual medical devices. In some embodiments, the auxiliary monitor includes at least one processor configured to process the data received from the multiple medical devices. In some instances, the data from multiple medical devices may be combined in a way that is not possible when the data from each medical device is considered in isolation.

The inventors have further recognized and appreciated that controllers of medical devices, such as cardiac support systems (e.g., controller 130 of heart pump 110), typically have limited functionality and processing power, which may be tailored to efficiently monitor and control operation of the devices. To this end, some embodiments of the technology described herein may extend the capabilities of such controllers by, for example, including an auxiliary monitor with additional processing capabilities and/or storage capacity for determining metrics (e.g., advanced metrics using one or more machine learning (ML) or artificial intelligence (AI) techniques) using data received from one or more controllers or other devices.

As described in further detail herein, the inventors have also recognized that some healthcare providers (e.g., surgeons or interventionalists) are typically unable to monitor some metrics displayed on the controller of a cardiac support system during a procedure due, at least in part, to the display having limited real estate, visibility, and/or access to the healthcare provider during the procedure. Some embodiments of the present disclosure enable the healthcare provider to view one or more metrics associated with operation of one or more medical devices without having to interact with or view the displays of the controllers associated with each individual device.

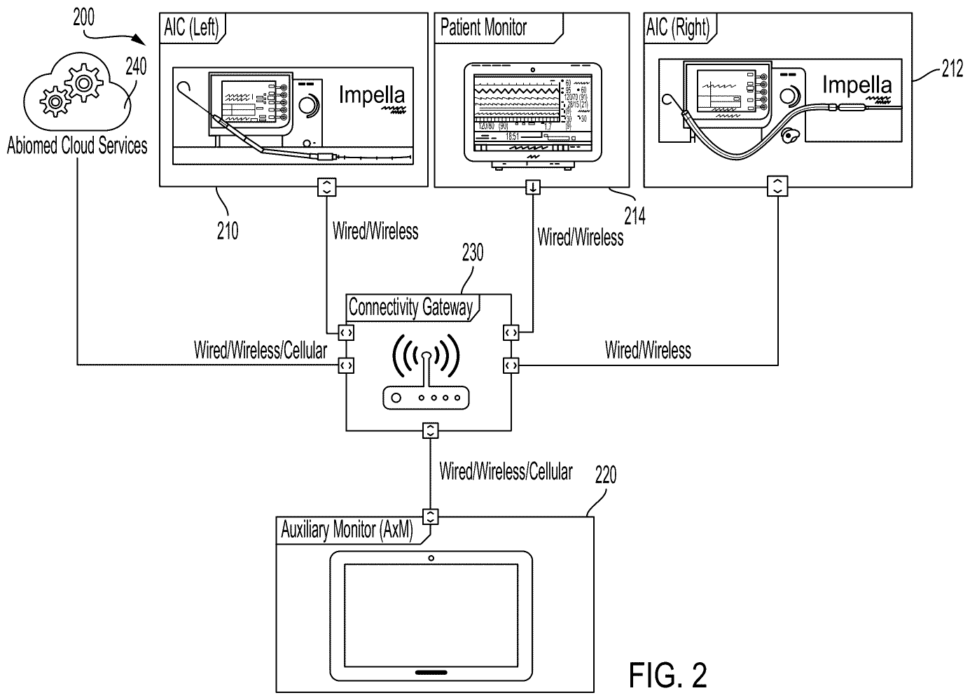

FIG. 2 illustrates a patient monitoring system 200 in accordance with some embodiments of the present disclosure. The patient monitoring system 200 includes a plurality of patient monitoring devices (e.g., AICs 210 and 212, and patient monitor 214) coupled to an auxiliary monitor 220 via a connectivity gateway 230. As will be appreciated, any suitable patient monitoring devices may be included in patient monitoring system 200. For instance, patient monitoring system may be implemented as a heart recovery system that includes one or more cardiac or cardio-pulmonary support consoles (e.g., an AIC, a blood oxygenation system, a balloon catheter and pump controller device) connected to connectivity gateway 230. Connectivity gateway 230 may provide a networking interface to transfer data between the patient monitoring devices and auxiliary monitor 220. As shown, the network connections between the patient monitoring devices and connectivity gateway may include wired (e.g., Ethernet, USB, coaxial cable) and/or wireless (e.g., WiFi, Cellular, Bluetooth) network interfaces. Data transmission may be achieved using any suitable transmission protocol examples of which include TCP and UDP. The auxiliary monitor 220 may be configured to encode/decode streams of TCP/UDP data and perform localized computation using the received data. The patient monitoring devices may include one or more controllers of a cardiac support system (e.g., AIC (Left) 210, AIC (Right) 212) configured to monitor parameters associated with a heart pump (e.g., pump speed, flow rate, purge flow rate, purge pressure), one or more patient monitoring devices (e.g., patient monitor 214) configured to monitor patient vital signs (e.g., heart rate, blood oxygenation, blood pressure, respiratory rate), and/or one or more other patient monitoring devices.

Data transmission between the patient monitoring devices and connectivity gateway 230 may be unidirectional and/or bidirectional. For instance, connectivity gateway 230 may be configured to receive monitored information from AICs 210 and 212 and patient monitor 214 and pass the monitored information (with or without transformation) to auxiliary monitor 220. In other embodiments, at least one of the connections between the patient monitoring devices and connectivity gateway 230 may be bidirectional. For instance, auxiliary monitor 220 may be configured to issue a request to connectivity gateway 230 for particular data from one of the patient monitoring devices, and in response, the corresponding patient monitoring device may provide the requested data to connectivity gateway 230, which in turn may provide the requested data to auxiliary monitor 220 for processing and/or display.

As described herein, auxiliary monitor 220 may be configured to determine one or more hemodynamic signals, predict one or more hemodynamic signals, and/or evaluate whether an alert threshold has been met. The auxiliary monitor 220 may display indications of data received via the connectivity gateway in a configurable user interface using one or more waveforms, values, trends and/or indicators. In still other embodiments, the auxiliary monitor may be configured to display patient data, such as data received from an electronic health record (EHR) stored in a hospital information system.

In some embodiments, patient monitoring system 200 may be configured to segregate higher-risk control functions performed by the controller(s) for controlling operation of a connected device from lower-risk monitoring functions that collect and report data about the operation of the device and/or a physiological state of a patient associated with the device. By segregating control from monitoring, the auxiliary monitor 220 may be classified by the U.S. Food and Drug Administration (FDA) as a class II medical device (moderate risk) rather than a class III medical device (high risk). As such, software updates (e.g., new data processing algorithms, updates to data integration, etc.) for the auxiliary monitor 220 when classified as a class II medical device may not require as rigorous validation to ensure patient safety (e.g., through documentation provided to an approved by regulatory bodies) compared to devices classified as class III medical devices. The reduced requirements may allow for faster integration and/or distribution of new features in a clinical setting than if the updates were made directly to the controller(s). In some embodiments, updates to the auxiliary monitor 220 to include new features may be made remotely via connectivity gateway 230 as the features move through the verification and validation phase to further enhance rapid deployment of the new features.

As shown in FIG. 2, auxiliary monitor 220 may be configured to be coupled to multiple patient monitoring devices including, but not limited to, one or more AICs (e.g., AIC 210, 212), one or more patient monitors (e.g., patient monitor 214) or other patient monitoring devices.

As will be appreciated, although the heart pumps are shown as being connected to the AICs, which are thereafter connected to the auxiliary monitor 220, it will be appreciated that in other embodiments the heart pumps may be connected directly to an auxiliary monitor. In such embodiments, the auxiliary monitor 220 may be connected to a second auxiliary monitor (not shown), which may be configured to connect to multiple auxiliary monitors. In such embodiments, the second auxiliary monitor may be configured in a hub and spoke configuration such that it may be connected to multiple patients at the same time. For example, in one embodiment, a clinician (e.g., a doctor and/or a nurse) may use the second auxiliary monitor to monitor the support (e.g., via heart pumps and/or other devices) of multiple patients in the same hospital.

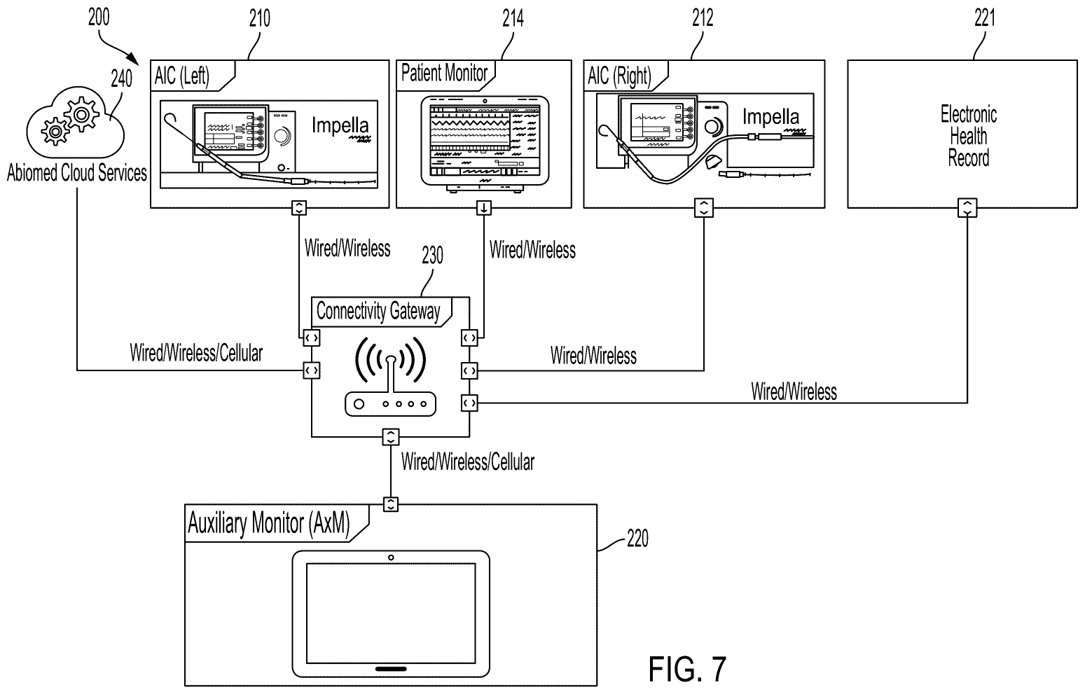

As shown in FIG. 7, in some embodiments, auxiliary monitor 220 may also be coupled to one or more additional information sources. For example, the auxiliary monitoring system may be connected to electronic health records (EHRs), 221 stored by a hospital information system. In such embodiments, the auxiliary monitor 220 may be connected to the EHR(s) 221 via the gateway 230, such as via a wireless or wired connection. In some embodiments, an EHR 221 may be configured to transmit patient data to the auxiliary monitor (e.g., via the gateway). In other embodiments, pump data may be transferred from one of the devices to the auxiliary monitor and thereafter to the EHR (e.g., for storage in a patient record). As will be appreciated and as described herein, appropriate security protocols may be in place to safeguard patient data being received and/or transferred to the EHR from the auxiliary monitor.

Each of the coupled patient monitoring devices and/or additional information sources may be associated with its own information stream that includes data corresponding to one or more monitored parameters associated with the corresponding device. In some embodiments, auxiliary monitor 220 may be configured to leverage multiple information streams received from the multiple devices and/or information sources to perform data processing that is not possible when only a single information stream is considered. In this way, the information provided and/or displayed by auxiliary monitor 220 may not be merely duplicative of that shown on the individual patient monitoring devices (e.g., AICs 210, 212, an ECMO device, etc.) or in an additional information source (e.g., the EHRs). Instead, information provided and/or displayed by the auxiliary monitor can include information determined, at least in part, on information from all or a subset of the coupled patient monitoring devices and/or additional information sources. In this regard, one or more of the algorithms disclosed herein may include data received from one or more of the patient monitoring devices and from one or more of the additional information sources (e.g., the EHR 221).

As described in more detail herein, a patient may have multiple cardiac support devices (e.g., a right system and a left system). Metrics that rely on information from both of the cardiac support devices may be determined and recommendations on how to modify operation of one or both of the cardiac support devices to optimize patient outcomes may be provided to guide treatment. For instance, a recommendation to increase or decrease support for one or both of the cardiac support devices may be determined, such as to maintain balanced support across both of the cardiac support devices. Algorithms for escalation or weaning may use one or more models that predict, based on current information from multiple information streams, how a change in the level of support may affect the cardiac and/or overall health of the patient (e.g., as the patient is working toward a particular goal, such as weaning off of a device). In this way, the auxiliary monitor 220 may be configured to provide a physician or other healthcare provider a more integrated or complete view of a patient's health than can be obtained from viewing multiple displays associated with individual patient monitoring devices.

In some embodiments, the auxiliary monitor 220 may be implemented as a desktop portable device or pole mountable unit. In some embodiments, the auxiliary monitor 220 may include a touch screen display that enables a healthcare professional to interact with the display to reconfigure what is displayed on the auxiliary monitor 220 as desired.

In some embodiments, the auxiliary monitor 220 may be configured to send one or more computed values via connectivity gateway 230 to a web-based service, such as cloud service 240. Cloud service 240 may be configured to re-display the computed values, or information derived based on the computed values, via a web-based interface (e.g., a web browser). As will be appreciated, in some embodiments, unprocessed data also may be sent directly to the cloud service. In some embodiments, the auxiliary monitor may be configured to store the data feed, such as if the auxiliary monitor were not connected to the Internet. In such embodiments, the auxiliary monitor may be configured to transfer the data to the cloud service when the auxiliary monitor is then again connected to the Internet.

In some embodiments, auxiliary monitor 220 may be configured for use as a sensor gateway. The sensor gateway may be configured to combine parameters for sensor fusion and/or to provide (e.g., stream) sensor fusion data to a web-based services (e.g., cloud service 240). For instance, in some embodiments auxiliary monitor 220 may send data to another computing device (not shown) configured as a sensor gateway. The sensor gateway may, in turn, provide sensor fusion data to one or more other devices via, for example, connectivity gateway 230.

In some embodiments, auxiliary monitor 220 may be configured to be coupled to one or more additional devices. Such additional devices may provide information that relates to and/or may be informed by context of hemodynamics support devices (e.g., one or more heart pumps). Non-limiting examples of such additional devices include infusion pumps, ventilators, ECMO machines, dialysis machines, devices for point-of-care blood measurements, imaging devices for point-of-care imaging (e.g., echo or ultrasound devices), and/or monitoring patches. In some embodiments, the additional devices coupled to auxiliary monitor 220 may provide high-fidelity, continuous signals from one or more physiological measurements or therapies. The continuous signals from such devices may provide context to the measurements, position, and/or algorithms used by a hemodynamic support device, such as a heart pump. For example, in an illustrative embodiment in which an imaging device (e.g., an echo device) is connected to the auxiliary monitor, the auxiliary monitor may be used to view an image of the patient's heart to confirm and/or adjust the position of a hemodynamic device (e.g., a percutaneous pump) in the patient. In this example, the imaging device may be used by a clinician after an alarm is received, as described herein, such as a pump malfunction alarm, to allow the physician to confirm and/or adjust a position of the pump to improve patient support. Additionally, data from a hemodynamics support device may provide context to measurements and/or therapies of one or more of the additional devices coupled to the auxiliary monitor 220.

In other embodiments, the auxiliary monitor may be configured to monitor and process data from the one or more cardiac support devices and the one or more additional devices and to tailor support of the one or more cardiac support devise and/or the one or more additional devices such that the support across the devices may be balanced.

Some exemplary advantages of a patient monitoring system configured in accordance with the technology described herein include, but are not limited to, enabling advanced metrics or indicators from patient monitoring devices to be displayed in a surgical and interventional setting, enabling surgeons or interventionalists to view cardiac catheter data and advanced metrics on a secondary screen when the primary screen is typically inaccessible to them. Such a system may provide a platform for integrating metrics from other instruments or devices into a single display, provide a platform that can maintain a case context across multiple devices (e.g., maintain a history following a pump swap for escalation of support), and/or provide a platform to consolidate data for single or multiple patients.

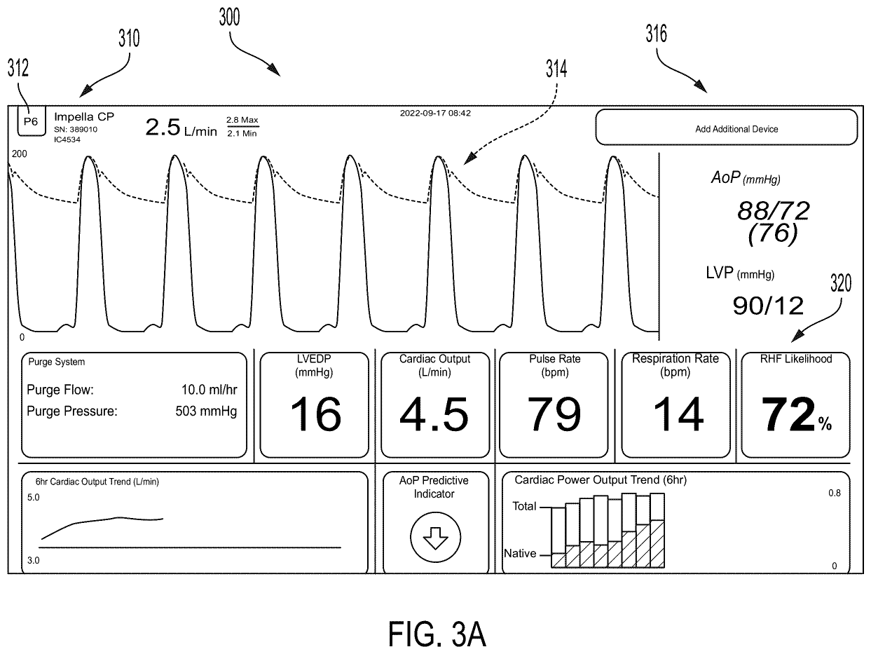

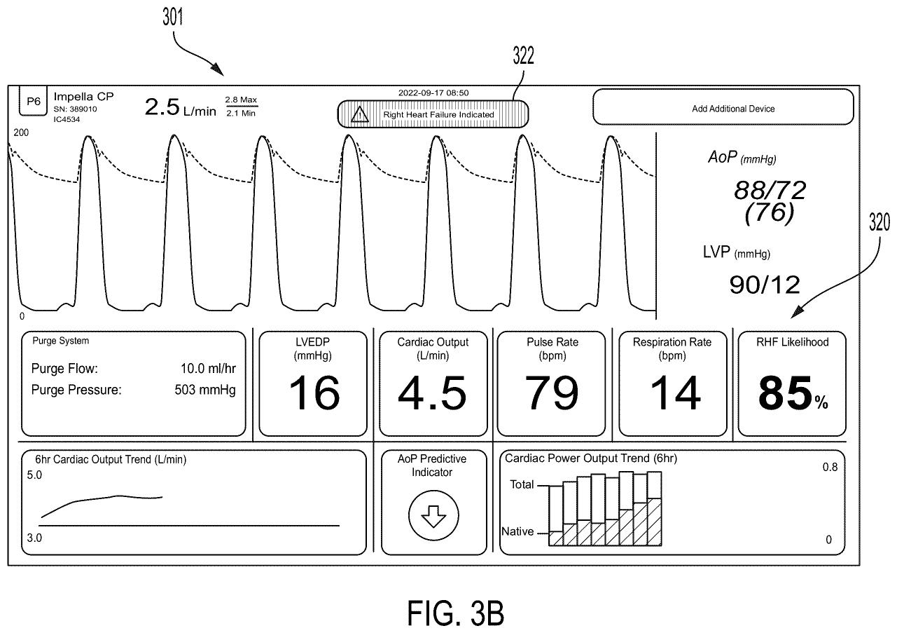

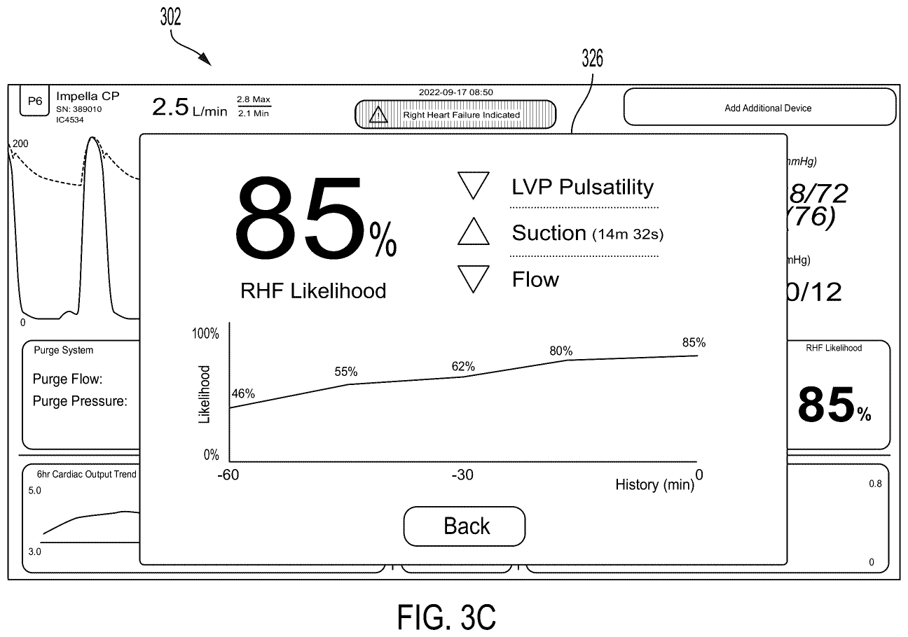

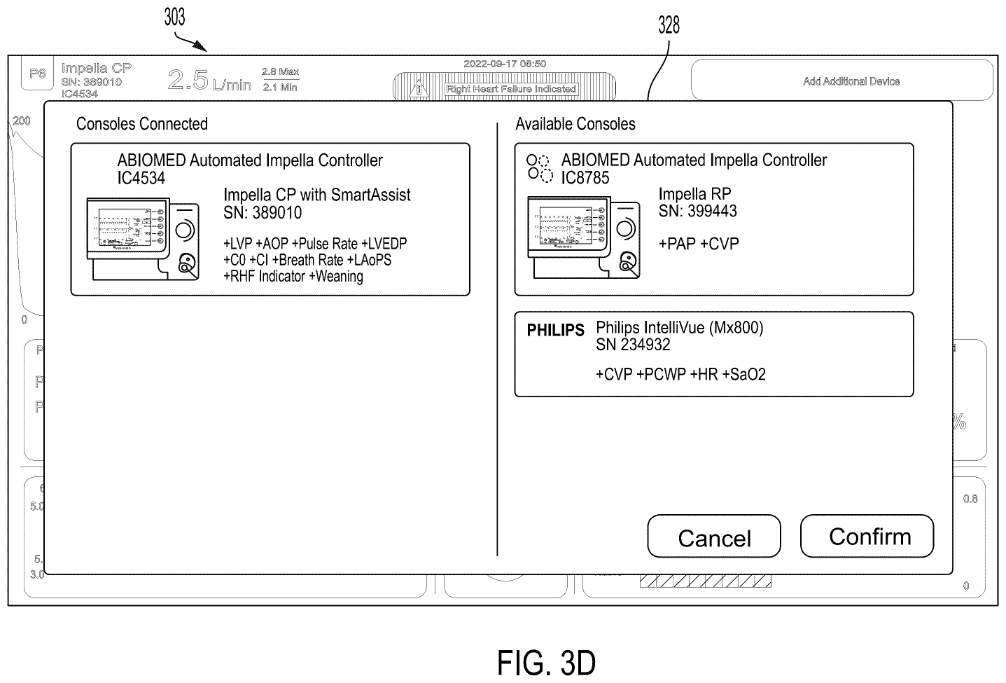

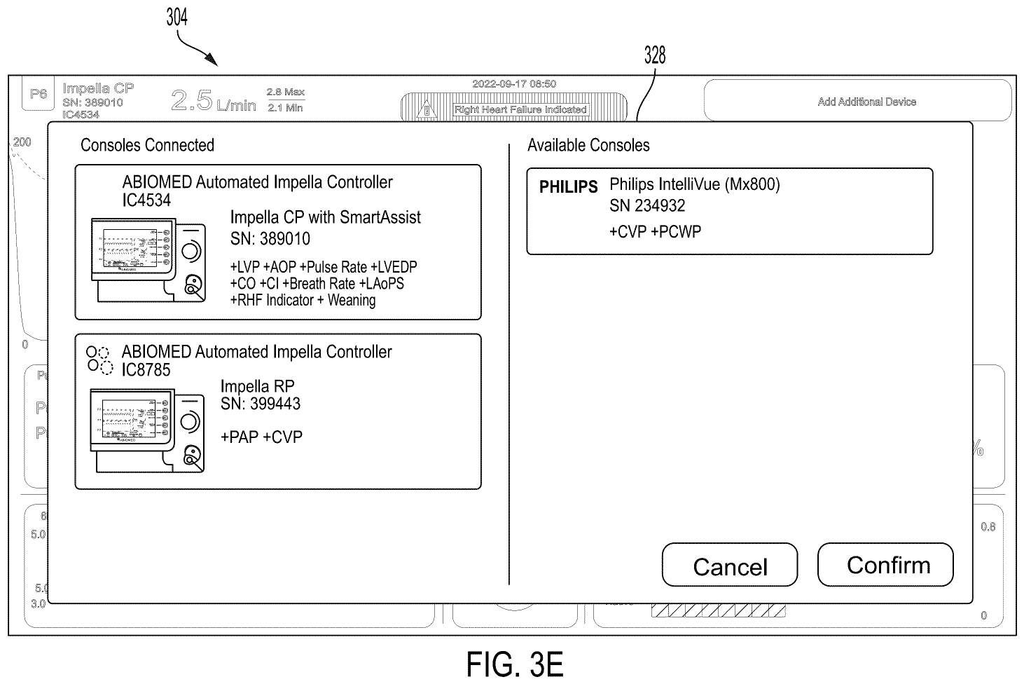

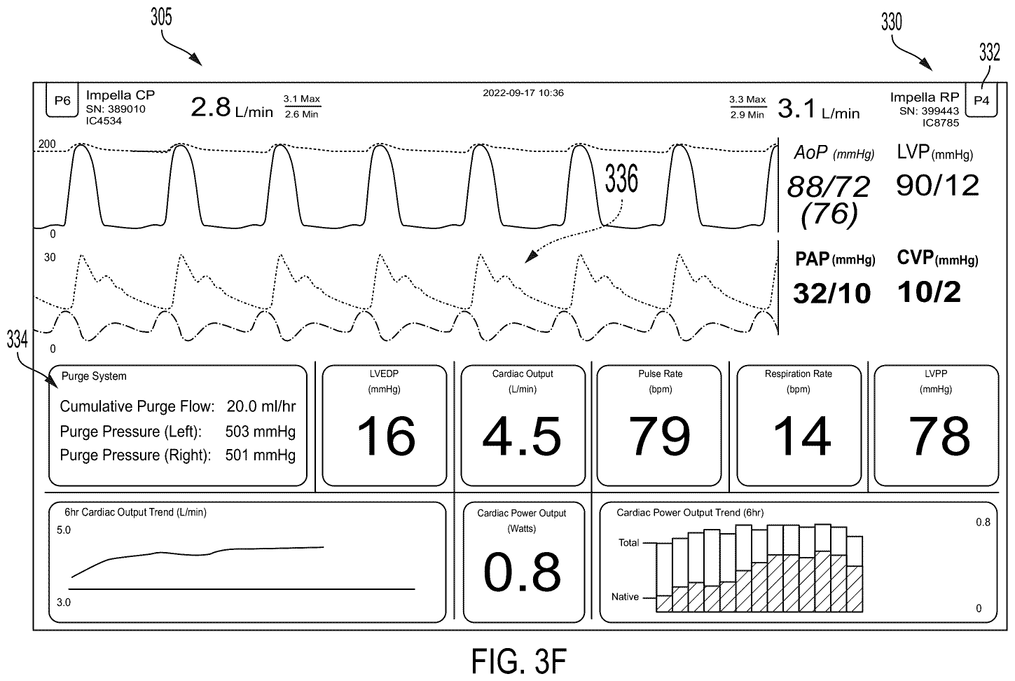

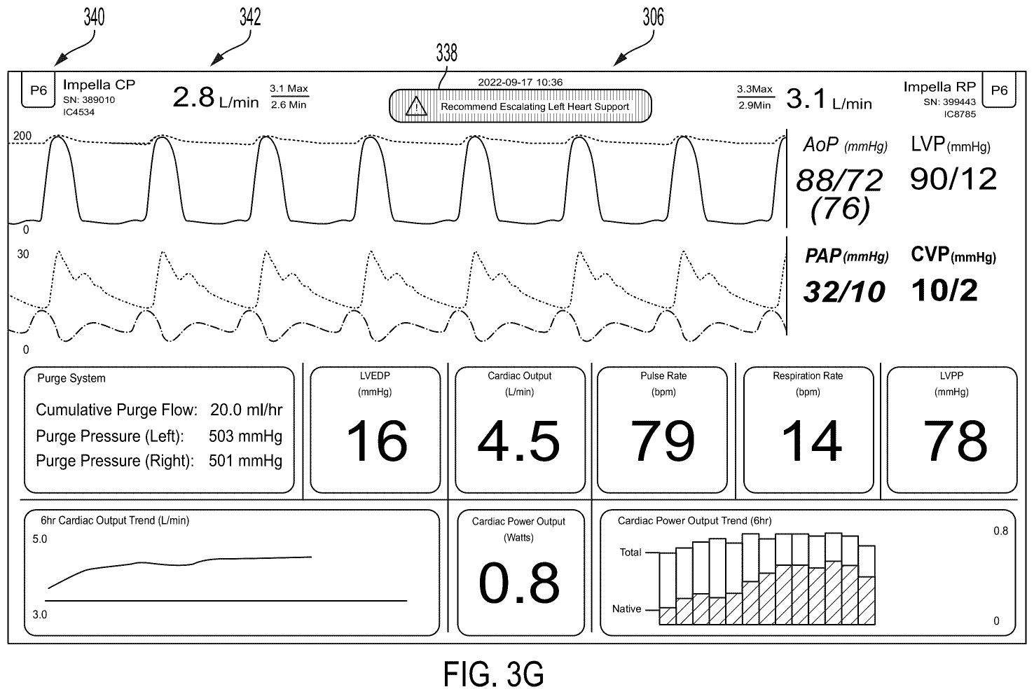

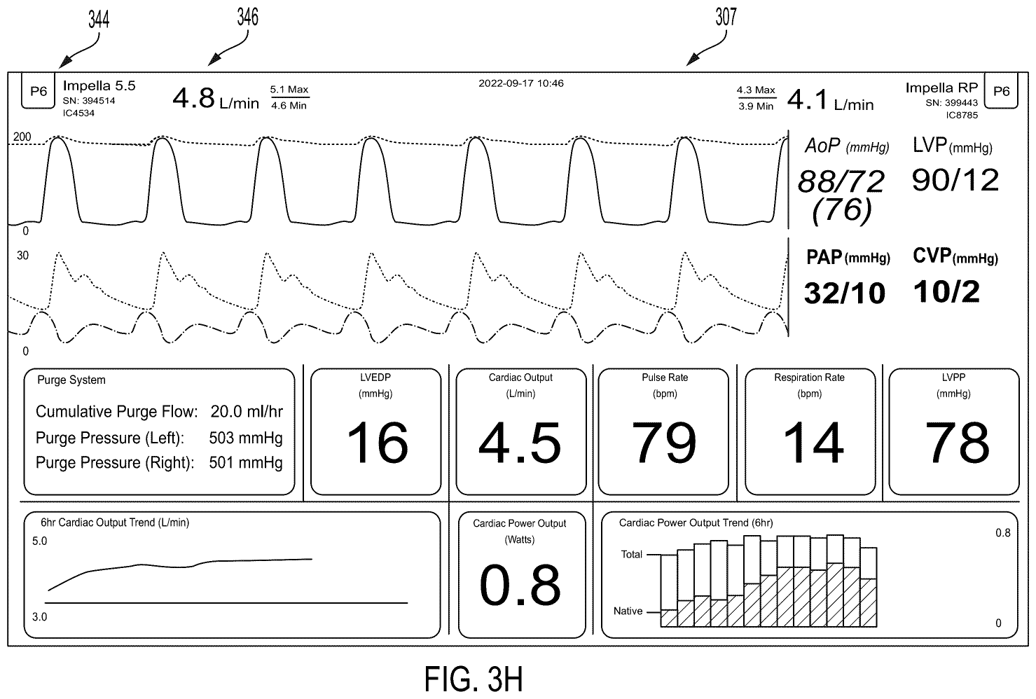

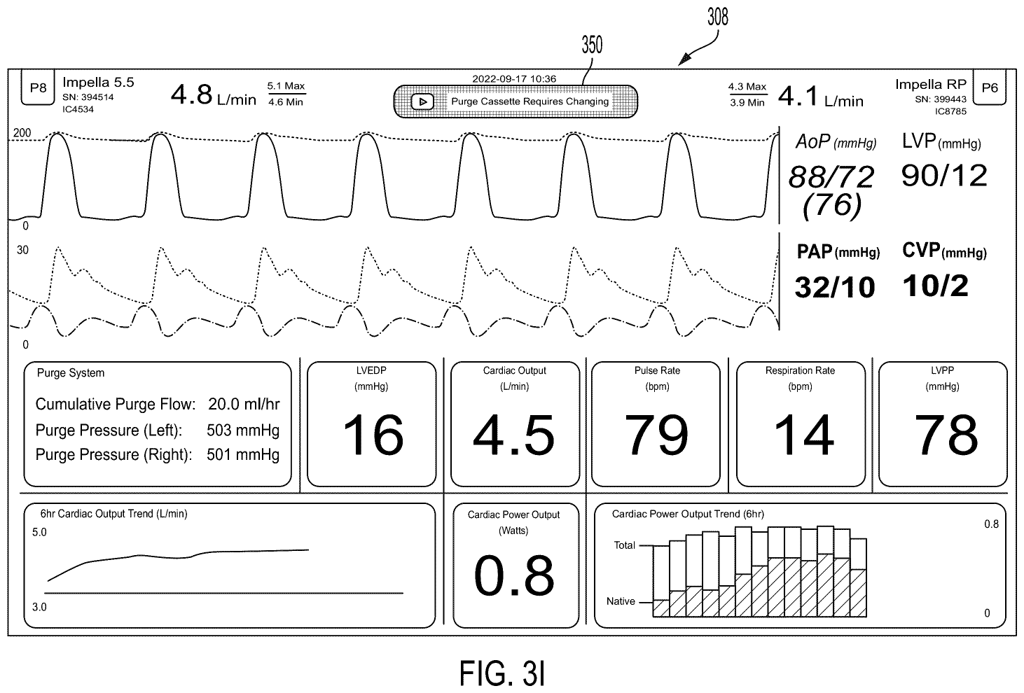

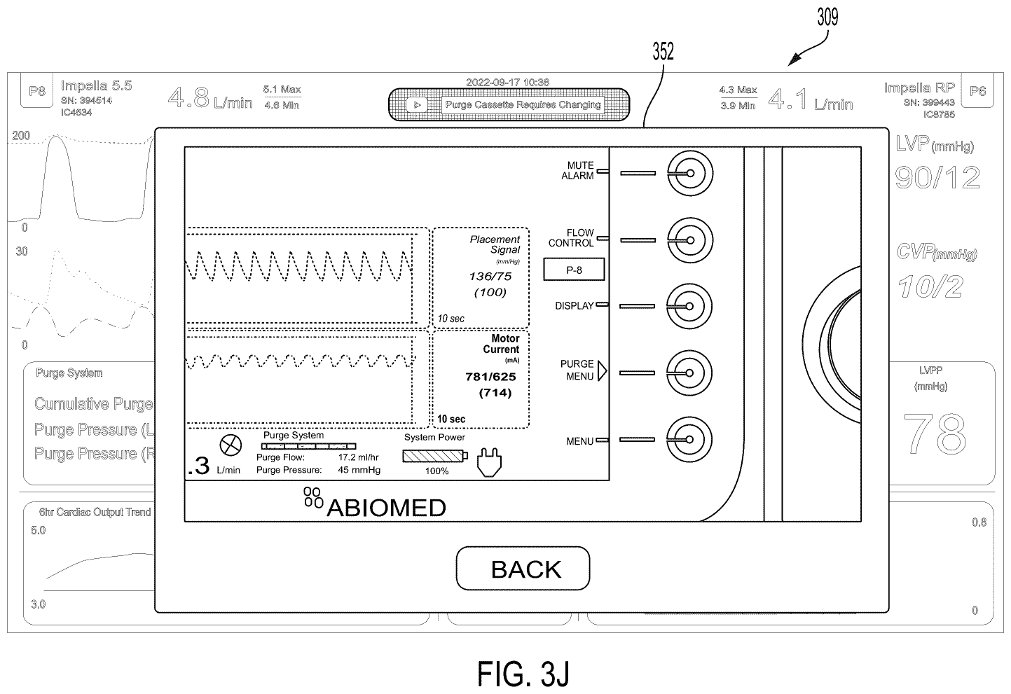

FIGS. 3A-3J illustrate exemplary portions of a user interface for auxiliary monitor 220 in accordance with some embodiments of the present disclosure. FIG. 3A illustrates a user interface 300 in which monitored information from a single AIC is displayed. As shown, user interface 300 may include a name 310 for the AIC and a speed 312 at which the AIC is currently operating. User interface 300 may further include one or more graphs 314 configured to show monitored metrics over time. For instance, as shown in FIG. 3A, aortic pressure (AoP) and left ventricular pressure LVP may be monitored and user interface 300 may be configured to display for each of these metrics, both a numerical value and a graph showing changes in the metric over time. In the example shown in FIG. 3A, the auxiliary monitor may be configured to determine a likelihood of right heart failure (RHF likelihood 320) based, at least in part, on the data received from the coupled devices (e.g., a cardiac support device and a patient monitor). As shown in FIG. 3A, the RHJF likelihood 320 is determined to be 72% in this example. Derived metrics such as RHF likelihood 320 may be determined in any suitable way. For instance, data received from one or more of the coupled devices may be provided as input to a trained machine learning model, and the output of the machine learning model may include the likelihood that the patient will have right heart failure (or some other derived metric). In some embodiments, an auxiliary monitor may be configured to process the data received from one or more the coupled devices using one or more stored algorithms to determine the derived data.

FIG. 3B illustrates a user interface 301, which is similar to user interface 300, but additionally includes an alert 322, which has been generated based on the data received from the coupled devices. As shown in FIG. 3B, the RHIF likelihood 320 determined by the auxiliary monitor has increased to 85% from 72% as shown in the exemplary user interface 300 of FIG. 3A. In response to the increased value for RHIF likelihood 320, alert 322 may be presented in user interface 301 alerting a medical professional viewing the user interface 301 that remedial action may be recommended to prevent or reduce the risk of right heart failure of the patient.

FIG. 3C illustrates a user interface 302, in which an alert overlay 326 may provide additional details regarding the alert 322 generated and shown in FIG. 3B. For instance, as shown in alert overlay 326, the RHIF likelihood of 85% was determined based on a decrease in left ventricular pressure (LVP) pulsatility, an increase in suction and a decrease in blood flow. Alert overlay 326 may show a historical trend for the RHIF likelihood 320 over the previous hour (or some other timeframe). Such information may assist a physician or other healthcare provider to determine trends in derived measures, which may inform a choice of medical intervention.

As will be appreciated, although FIGS. 3B and 3C show an example in which an alert and alert overlay provide details regarding RHF, other alerts and corresponding alert overlays may be utilized in other embodiments.

In some embodiments, the user interface displayed on an auxiliary monitor may be configurable. For instance, the user interface may be configurable to suit different settings (e.g., catheterization lab, operating room, intensive care unit, nursing station, etc.) and/or to suit the individual preferences of healthcare practitioners. FIG. 3D illustrates a user interface 303 including a selection overlay 328. A user may interact with selection overlay 328 to configure the display of information on the user interface 303. Selection overlay 328 may be configured to display patient monitoring devices that are currently connected to the auxiliary monitor and available patient monitoring devices that may be connected to the auxiliary monitor. It should be appreciated that an “available” patient monitoring device shown in selection overlay 328 may be communicatively coupled to the auxiliary monitor (e.g., via connectivity gateway shown in FIG. 2), but may not be considered “connected” to the auxiliary monitor unless data from the patient monitoring device is being used to update the user interface of the auxiliary monitor. In the example shown in FIG. 3D, a first (e.g., left) cardiac support device (e.g., a first AIC) is currently connected to the auxiliary monitor, and a second (e.g., right) cardiac support device (e.g., a second AIC) and a bedside patient monitor are shown as being available for connection to the auxiliary monitor. A user of the auxiliary monitor may interact with selection overlay 328 of the user interface 303 to modify which patient monitoring device(s) are currently connected to the auxiliary monitor. For instance, the display of the auxiliary monitor on which the user interface is presented may be a touchscreen display that enables a user to drag and drop available patient monitoring devices into the connected portion of the selection overlay 328 (or vice versa). FIG. 3E illustrates a user interface 304, in which a user has interacted with selection overlay 328 to change a configuration of the user interface such that two cardiac support devices are selected as being connected and a patient monitoring device is shown as being available to connect. After modifying which patent monitoring devices are currently connected to the auxiliary monitor, the user may select the confirm button to implement the user's selection.

FIG. 3F illustrates a user interface 305 of an auxiliary monitor, which has been updated to display an indication of data received from multiple patient monitoring devices. As shown in FIG. 3F, in addition to displaying an indication of data received from a first patient monitoring device (e.g., a first AIC), user interface 305 may be configured to also display an indication of data received from a second patient monitoring device (e.g., a second AIC) when both patient monitoring devices are selected as being connected to the auxiliary monitor (e.g., as shown in FIG. 3E). User interface 305 may include a name 330 for the second AIC and a speed 332 at which the second AIC is currently operating. User interface 305 may further include one or more graphs 336 configured to show monitored metrics associated with the second AIC over time. For instance, as shown in FIG. 3F, pulmonary artery pressure (PAP) and central venous pressure (CVP) are being monitored and user interface 305 is configured to display for each of these metrics, both a numerical value and a graph showing changes in the metric over time.

In some embodiments, a user interface displayed on an auxiliary monitor may be configured to display one or more metrics that combine data received from multiple patient monitoring devices. For instance, as shown in FIG. 3F, a combined metric 334 showing cumulative purge flow across both of the connected AICs may be displayed on user interface 305. Other combined metrics 334 are also contemplated and embodiments of the present disclosure are not limited in this respect.

Calculating and displaying data combined from multiple patient monitoring devices may enable some embodiments to present a healthcare provider with information about a patient (or multiple patients) that is not possible when individual patient monitoring displays are used. In some embodiments, an auxiliary monitor may be configured to process data from multiple connected patient monitoring devices to generate alerts and/or assessments of a patient's health state that are not possible when only data from a single patient monitoring device is considered. FIG. 3G illustrates a user interface 306 having displayed thereon an alert 338 determined based, at least in part, on data received from a first cardiac support device and a second cardiac support device connected to an auxiliary monitor on which the user interface 306 is presented. As shown in FIG. 3G, the user interface 306 includes a pump speed (P6) 340 of the first (left) cardiac support device and a flow rate 342 of the first cardiac support device. The alert 338 recommends that the left heart support (i.e., provided by the first cardiac support device) be escalated.

FIG. 3H illustrates a user interface 307 after the pump speed of the first cardiac support device has been increased to provide additional left heart support. As shown in FIG. 3H, the pump speed 344 of the first cardiac support device has been increased from P6 to P8, resulting in an increased flow rate 346 through the first cardiac support device from 2.8 L/min to 4.8 L/min. In the example shown in FIGS. 3G and 3H, the pump speed of the second cardiac support device has also been increased from P4 to P6, resulting in an increased flow rate through the second cardiac support device from 3.1 L/min to 4.1 L/min. In response to the escalated left heart support, FIG. 3H shows that the alert 388 displayed in user interface 306 has been removed in user interface 307. In this way, a user interface of an auxiliary monitor may be dynamically updated using the techniques described herein to provide a current view of a health status of a patient associated with multiple patient monitoring devices.

Although the alert 388 displayed in user interface 306 is related to patient characteristics based on the connected patient monitor devices, it should be appreciated that other types of alerts may also be determined and displayed on a user interface of an auxiliary monitor in some embodiments. FIG. 3I illustrates a user interface 308 in which an alert 350 indicates that a purge cassette of a cardiac support device should be changed. Such an alert may be generated based, at least in part, on purge system data received from the first and/or second connected cardiac support devices.

FIG. 3J illustrates a user interface 309 including a device overlay 352, configured to enable a user to learn further details regarding an alert displayed on a user interface of an auxiliary monitor and/or steps the user may take to troubleshoot and/or address the alert. In the example of FIG. 3J, device overlay 352 may show a portion of an AIC display corresponding to the left cardiac support device connected to the auxiliary monitor. Further details about the purge system of the cardiac support device may be shown in the device overlay 352, which enable a user to troubleshoot and/or address the alert 350 shown in user interface 308 of FIG. 3I.

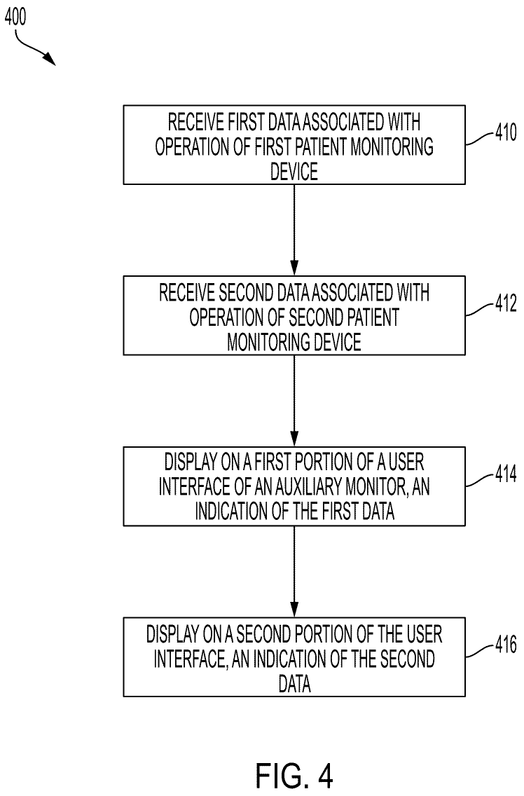

FIG. 4 is a flowchart of a process 400 for displaying, on a user interface of an auxiliary display, monitored data from multiple patient monitoring devices, in accordance with some embodiments. In act 410, first data associated with operation of a first patient monitoring device is received (e.g., by an auxiliary monitor). As described herein, the first patient monitoring device may be a cardiac support device (e.g., an AIC associated with a heart pump system), a bedside patient monitor configured to monitor a patient's vital signs, a blood glucose monitor, a respiratory monitor, or some other patient monitoring device. The first data received from the first patient monitoring device may include physiological data (e.g., pulse rate) for a patient with which the first patient monitoring device is associated and/or device data (e.g., pump speed) associated with operation of the first patient monitoring device itself. Additionally, the first data may be measured directly by the first patient monitoring device or may be derived based on data measured by the first patient monitoring device. For instance, the first patient monitoring device may include one or more processors configured to process measured data and the first data may include the processed data.

Process 400 may then proceed to act 412, where second data associated with operation of a second patient monitoring device is received (e.g., by the auxiliary monitor). As described herein, the second patient monitoring device may be a cardiac support device (e.g., an AIC associated with a heart pump system), a bedside patient monitor configured to monitor a patient's vital signs, a blood glucose monitor, a respiratory monitor, or some other patient monitoring device. The second data received from the second patient monitoring device may include physiological data (e.g., pulse rate) for a patient with which the second patient monitoring device is associated and/or device data (e.g., pump speed) associated with operation of the second patient monitoring device itself. Additionally, the second data may be measured directly by the second patient monitoring device or may be derived based on data measured by the second patient monitoring device. For instance, the second patient monitoring device may include one or more processors configured to process measured data and the second data may include the processed data.

Process 400 may then proceed to act 414, where an indication of the first data may be displayed on a first portion of a user interface of an auxiliary monitor. The indication of the first data may take any suitable form including, but not limited to, one or more graphs, charts, numerical values, waveforms, trends and the like. In some embodiments, the indication of the first data may be a representation of the first data as received from the first patient monitoring device (e.g., a pulse rate value received from the first patient monitoring device). In some embodiments, the indication of the first data may be a processed version of the first data received from the first patient monitoring device. For instance, the first data may be provided as input to a trained machine learning model, and the output of the trained machine learning model may be displayed on the user interface as the indication of the first data.

Process 400 may then proceed to act 416, where an indication of the second data may be displayed on a second portion of a user interface of an auxiliary monitor. The indication of the second data may take any suitable form including, but not limited to, one or more graphs, charts, numerical values, waveforms, trends and the like. In some embodiments, the indication of the second data may be a representation of the second data as received from the second patient monitoring device (e.g., a pulse rate value received from the second patient monitoring device). In some embodiments, the indication of the second data may be a processed version of the second data received from the first patient monitoring device. For instance, the second data may be provided as input to a trained machine learning model, and the output of the trained machine learning model may be displayed on the user interface as the indication of the second data. The indication of the first data and the indication of the second data may be displayed in the same form or a different form on the user interface. For instance, the indication of the first data may be displayed as one or more graphs in the first portion of the user interface and the indication of the second data may be displayed as one or more numerical values in the second portion of the user interface (see e.g., FIGS. 3A, 3B, and 3F).

The inventors have recognized and appreciated that an auxiliary monitor configured in accordance with the techniques described herein may present data to healthcare providers in a way that is not possible when patient monitoring devices are used in isolation to present monitored data. For instance, an auxiliary monitor configured to receive data from multiple patient monitoring devices may process the received data and generate new data determined based on the data from the multiple patient monitoring devices. The auxiliary monitor may be configured to implement other (e.g., computationally complex) algorithms that cannot be computed on controllers of the individual patient monitoring devices due to processing and/or storage limitations of those devices. Additionally, the auxiliary monitor may be configured to implement algorithms and/or computational models that take data from multiple devices as input to generate advanced metrics representing a health status of a patient. For instance, data from multiple patient monitoring devices may be used to determine a likelihood that the patient will have heart failure if remedial actions are not taken.

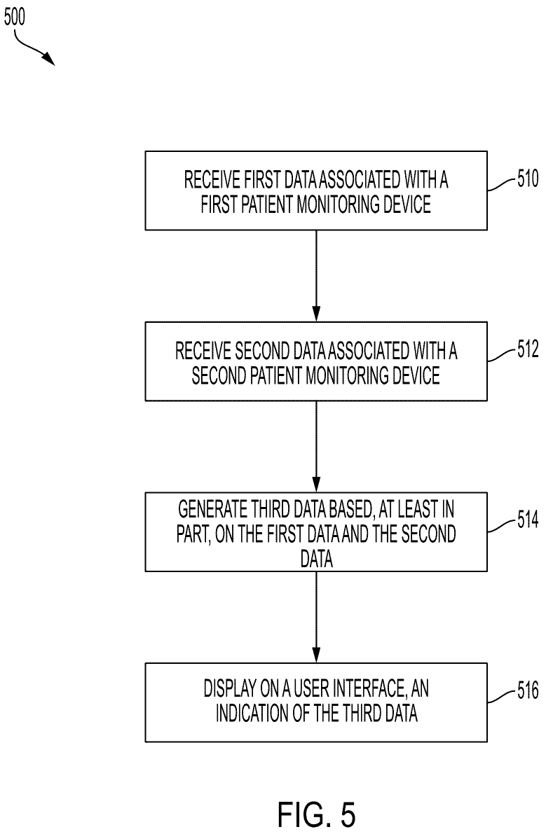

FIG. 5 is a process 500 for generating new data based, at least in part, on data received from multiple patient monitoring devices, in accordance with some embodiments. In act 510, first data may be received from a first patient monitoring device. Non-limiting examples of first data and first patient monitoring devices are described herein, for instance, with respect to act 410 of process 400. Process 500 may then proceed to act 512, where second data may be received from a second patient monitoring device. Non-limiting examples of second data and second patient monitoring devices are described herein, for instance, with respect to act 412 of process 400. Process 500 may then proceed to act 514, where third data may be generated based, at least in part, on the first data and the second data. The third data may be generated in any suitable way. For instance, as described herein, an auxiliary monitor configured to receive the first data and the second data may include and/or be associated with additional processing and/or storage resources that enable (e.g., computationally complex) processing of the first and second data which is not possible on the individual patient monitoring devices. As an example, the auxiliary monitor may be configured to provide the first data and the second data as input to one or more algorithms or models configured to output (as the third data) one or more predictions of how to transition the patient from a current health state to a desired heart state (e.g., weaning a patient off of a cardiac support device).

In some embodiments, the third data may represent a patient's health condition that cannot be assessed with the first data or the second data alone. For instance, a patient may be associated with a first (left) cardiac support device and a second (right) cardiac support device. The auxiliary monitor may be configured to receive first data from the first cardiac support device and second data from the second cardiac support device. Based on the first data and the second data an overall condition of the patient's heart may be assessed and monitored. In another example, a patient may be associated with a cardiac support device and a ECMO support device, with the auxiliary monitor configured to receive data from both devices and assess and monitor a condition of the patient (e.g., the condition of the heart of the patient).

In some embodiments, an auxiliary monitor may be communicatively coupled (e.g., using one or more networks) to one or more computational resources external to the auxiliary monitor. In such embodiments, the auxiliary monitor may be configured to send at least some of the first data and/or the second data to the external computational resource(s) for processing, and data returned from the external computational resource(s) may represent the third data generated in act 514.

Process 500 may then proceed to act 516, where an indication of the third data may be displayed on a user interface of the auxiliary monitor. The third data may be displayed in any suitable way. For instance, as shown in FIGS. 3A and 3B, a likelihood of heart failure determined from the first data and the second data may be displayed as a numerical value on the user interface. In some embodiments, the indication of the third data may include an alert displayed on the user interface, examples of which are shown in FIGS. 3G and 3I. An auxiliary monitor may be configured to implement one or more algorithms or models that take as input the first data and the second data to determine whether an alert should be generated, and when it is determined that an alert should be generated, the alert may be displayed on the user interface.

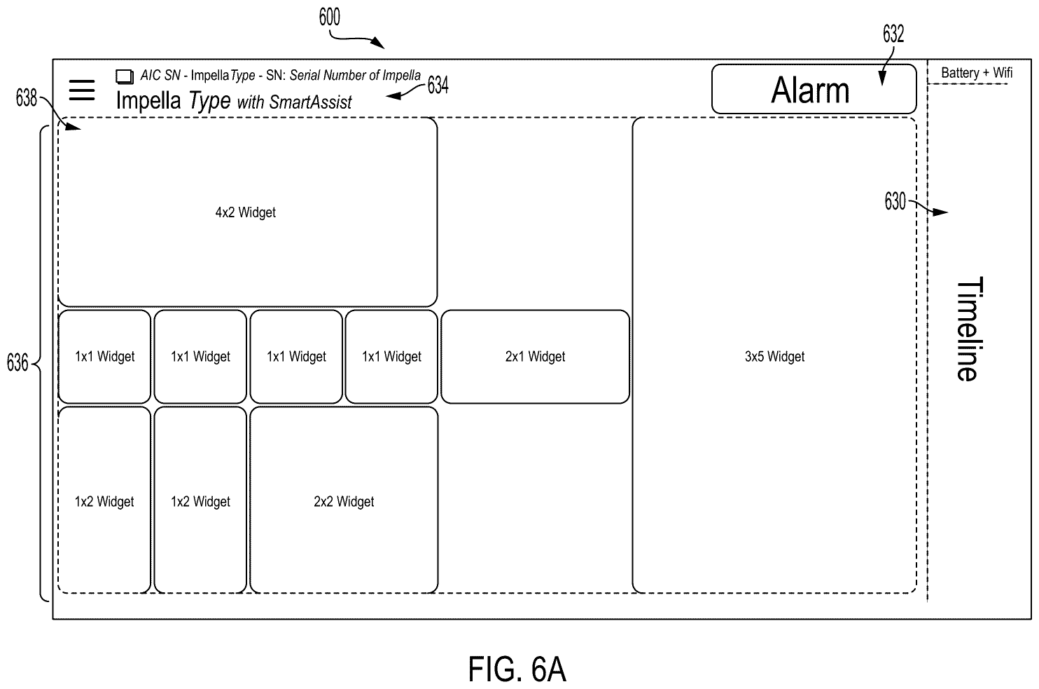

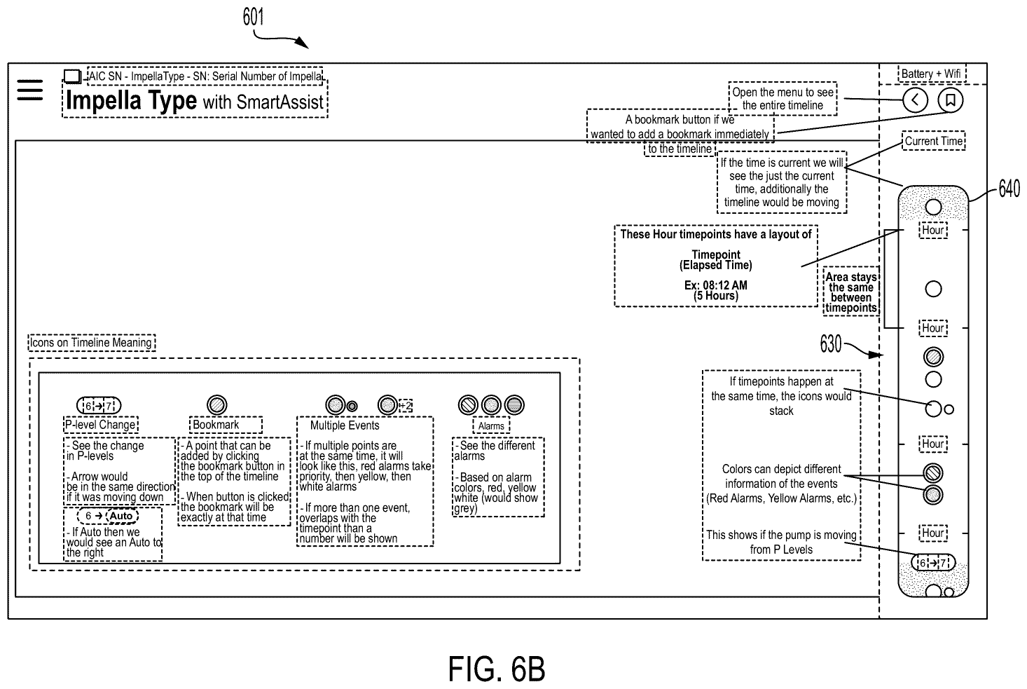

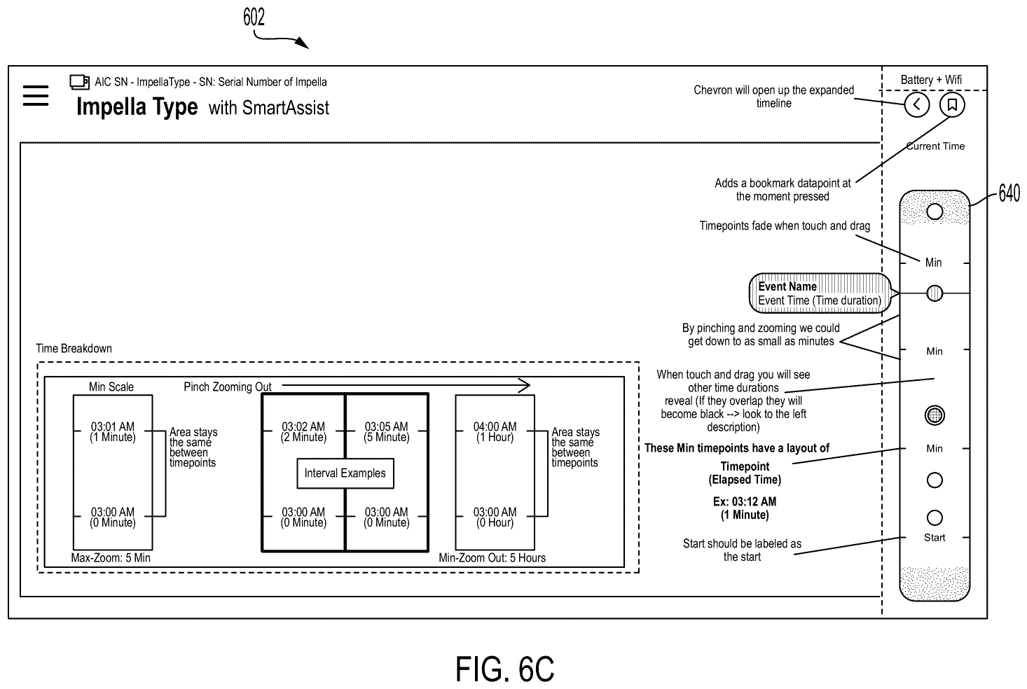

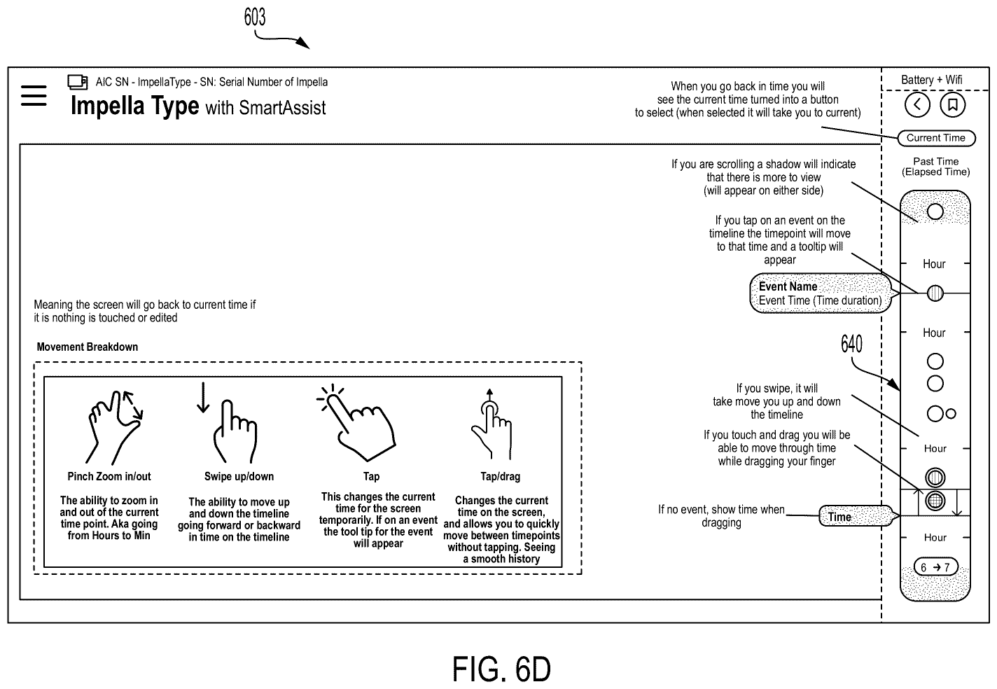

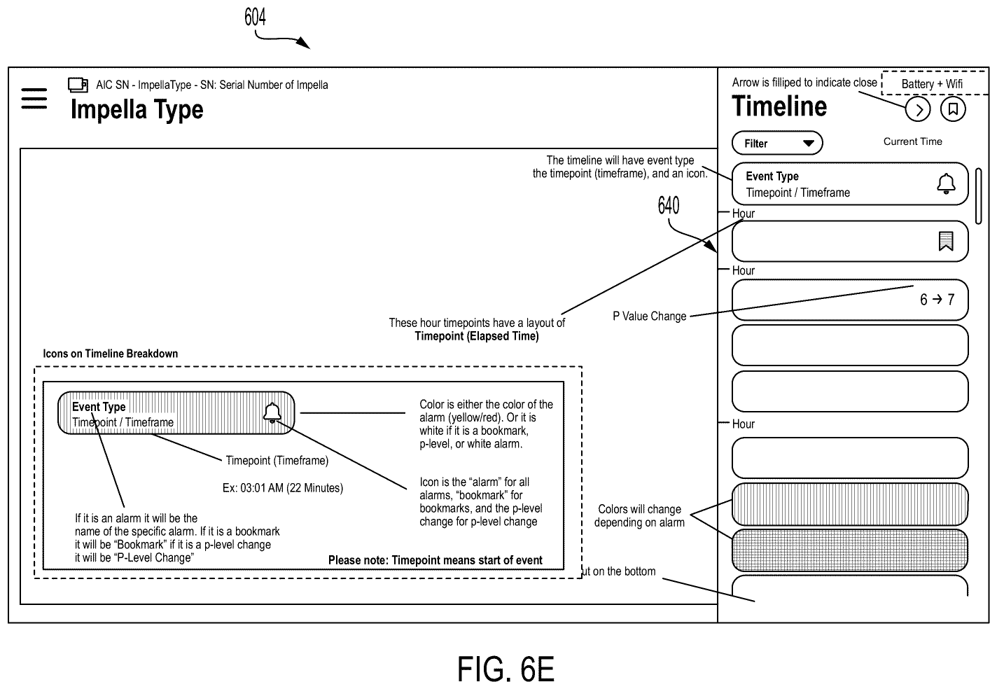

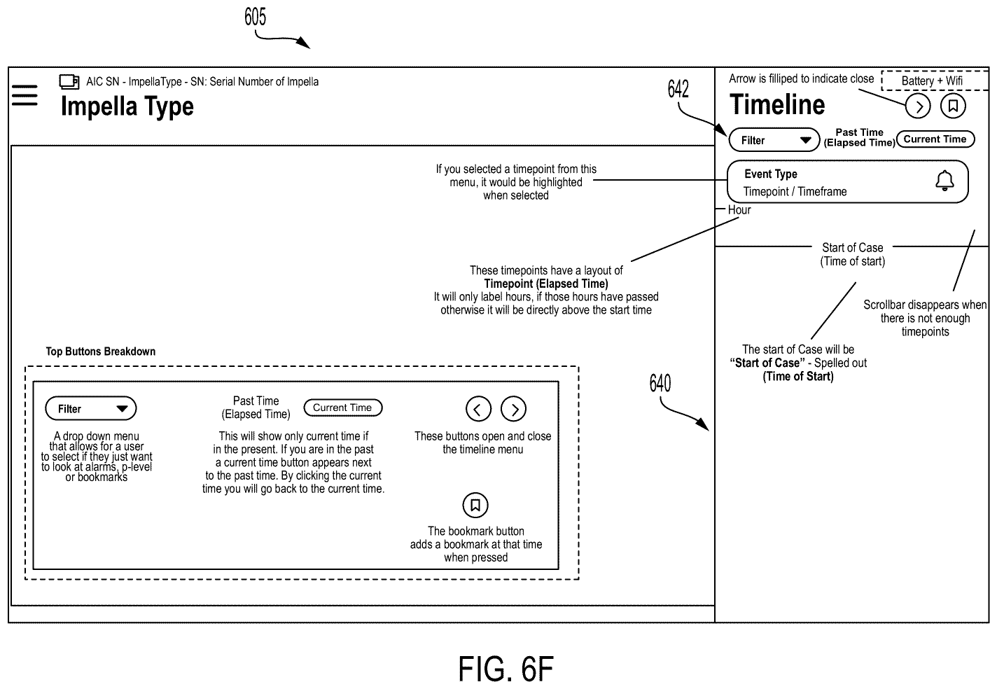

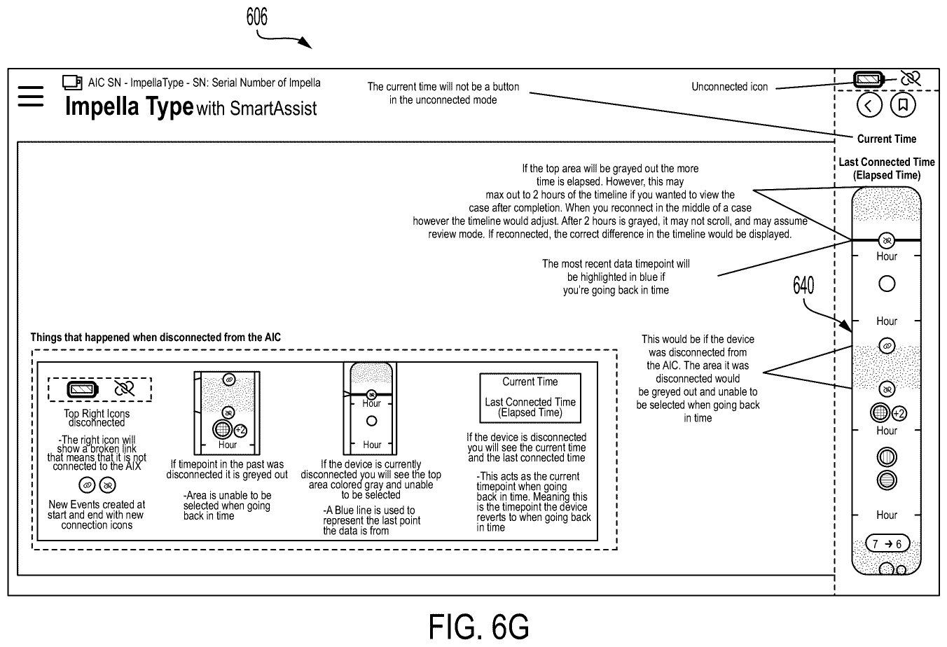

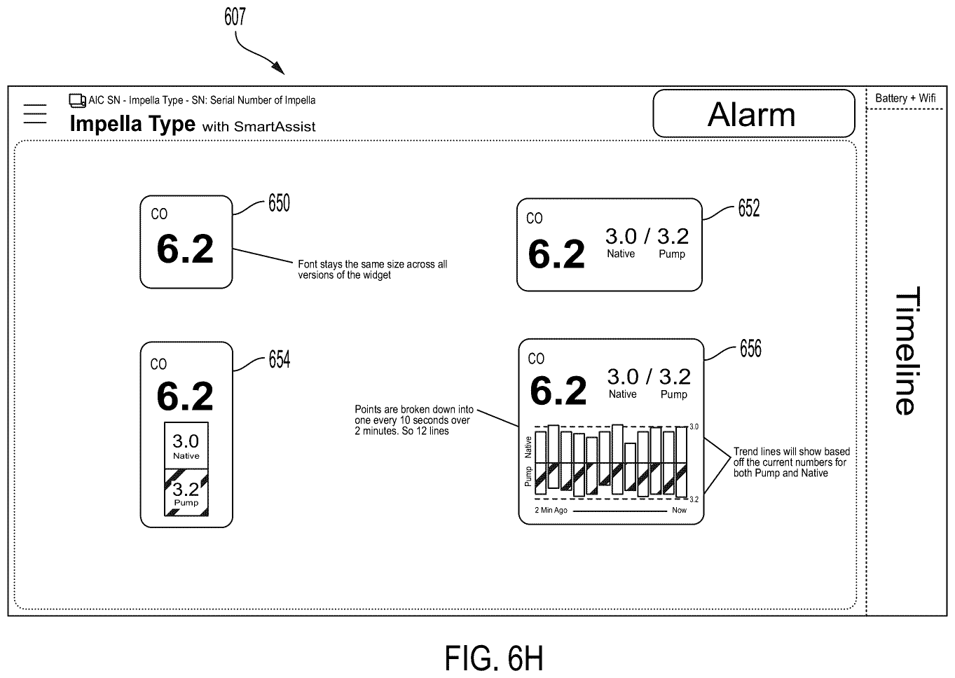

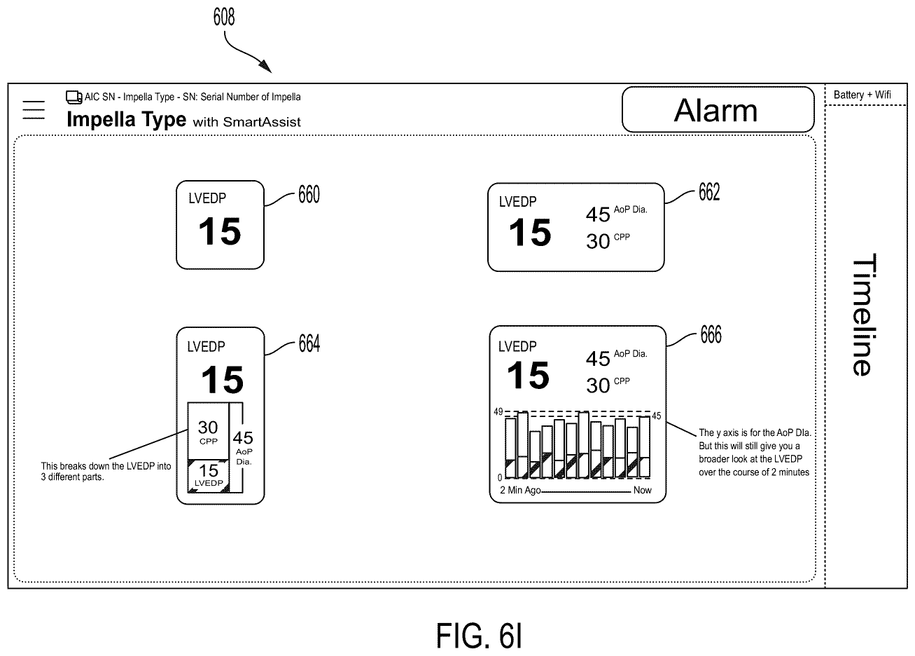

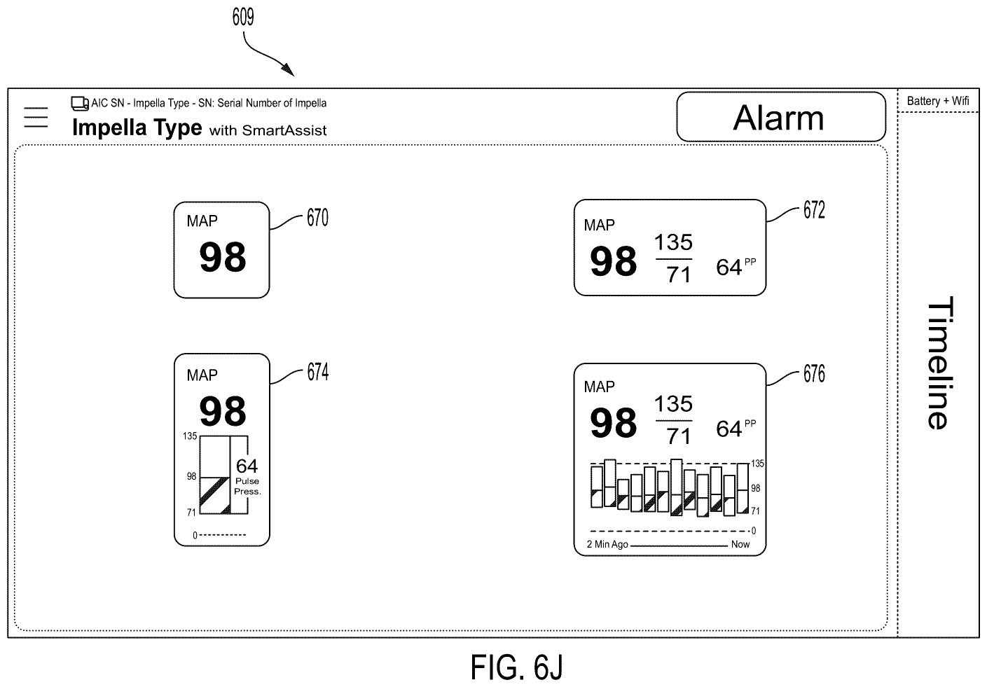

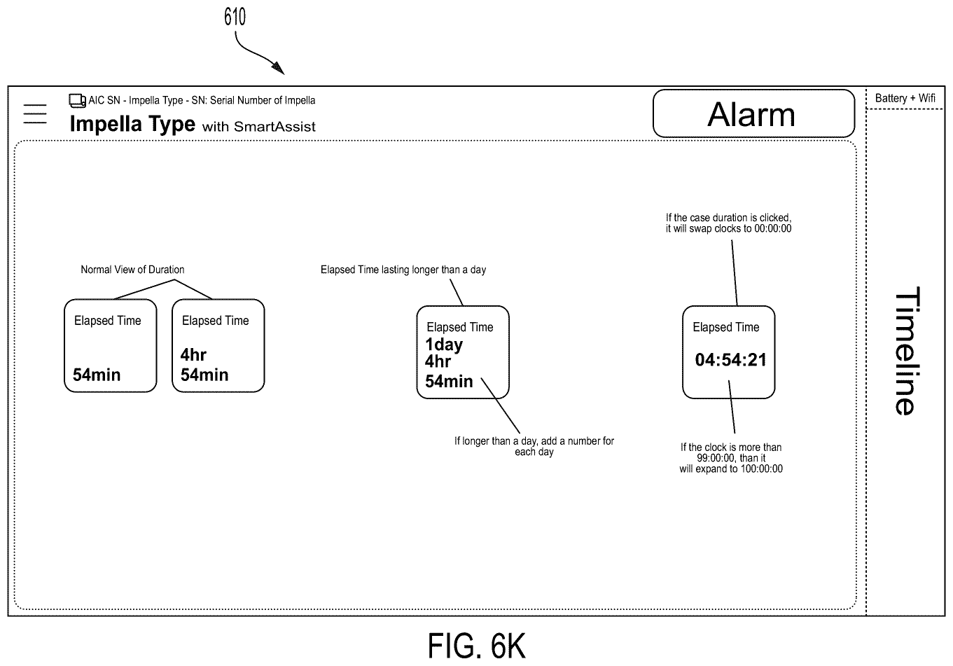

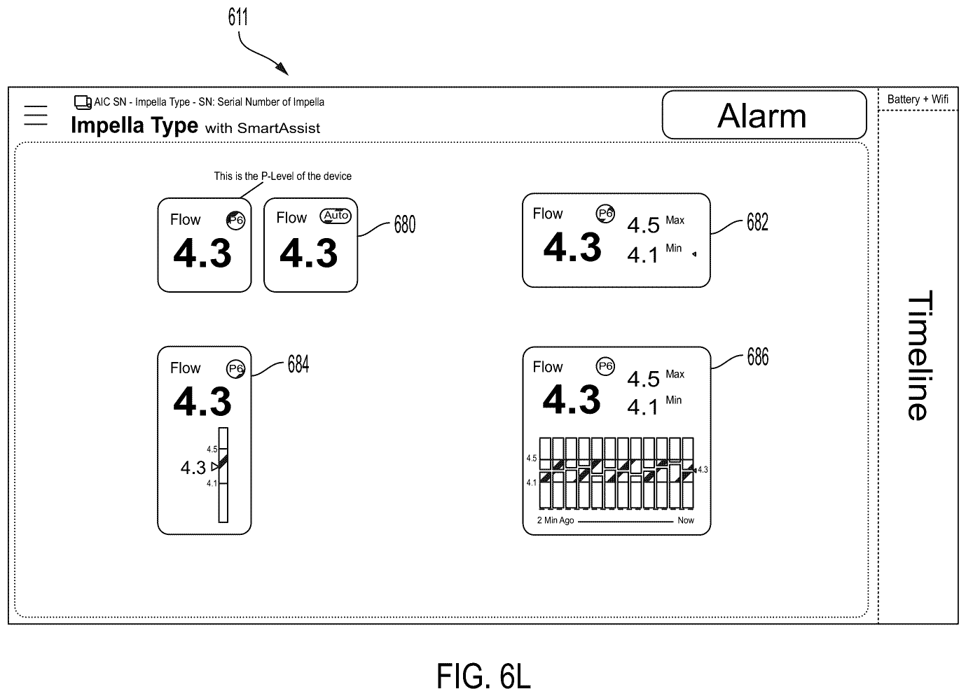

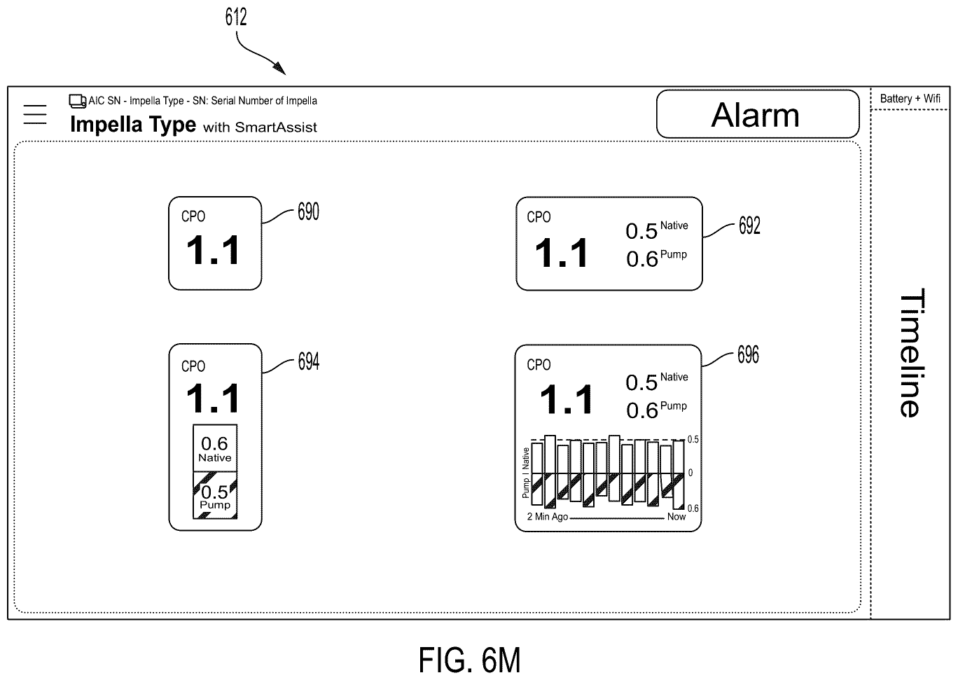

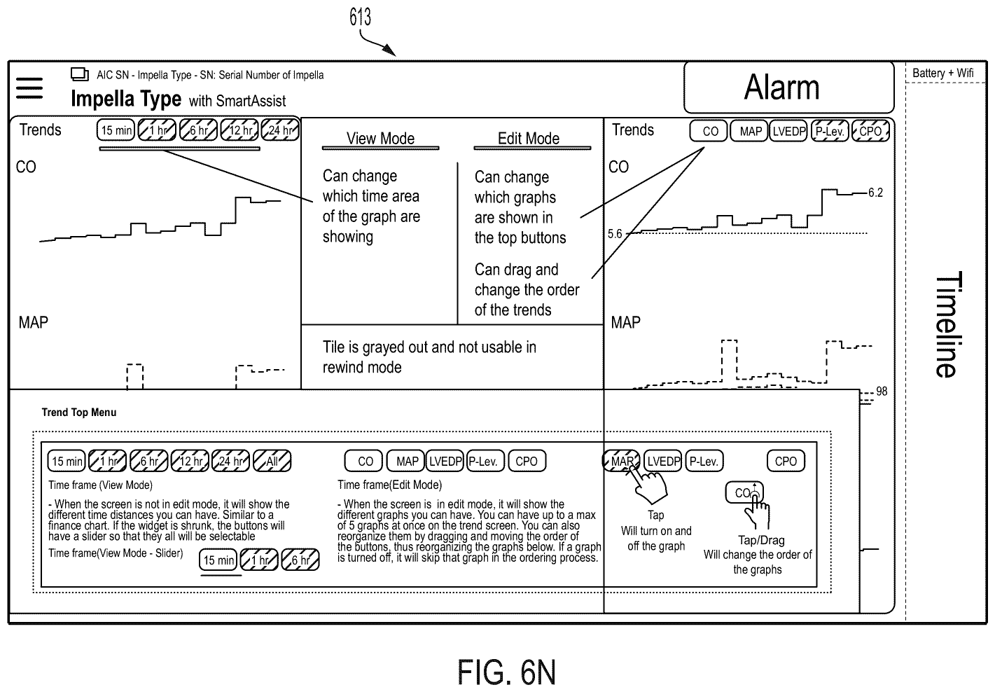

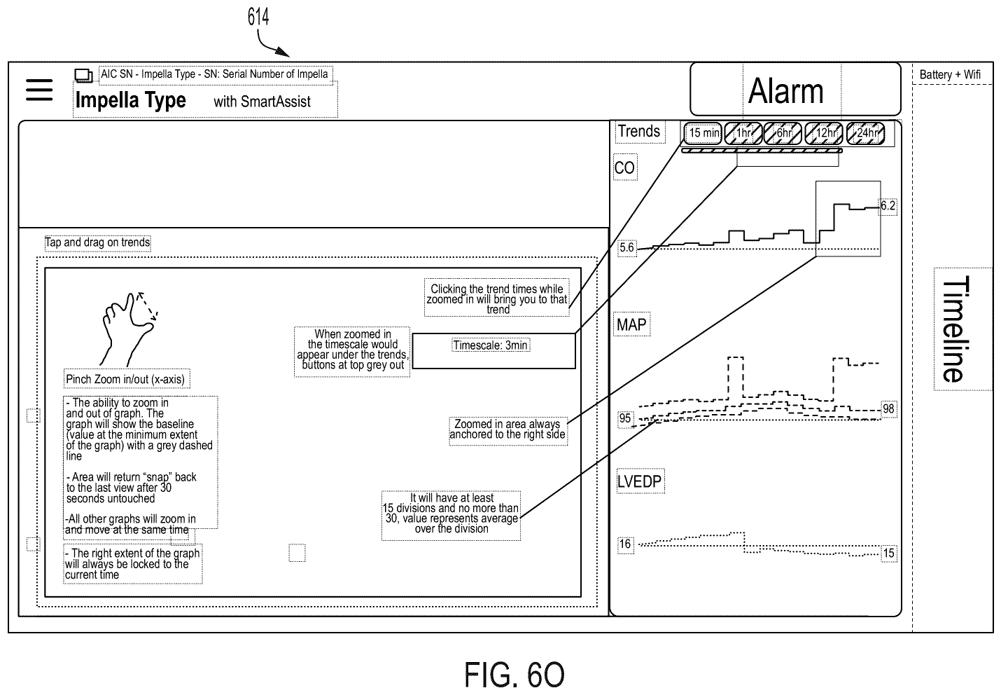

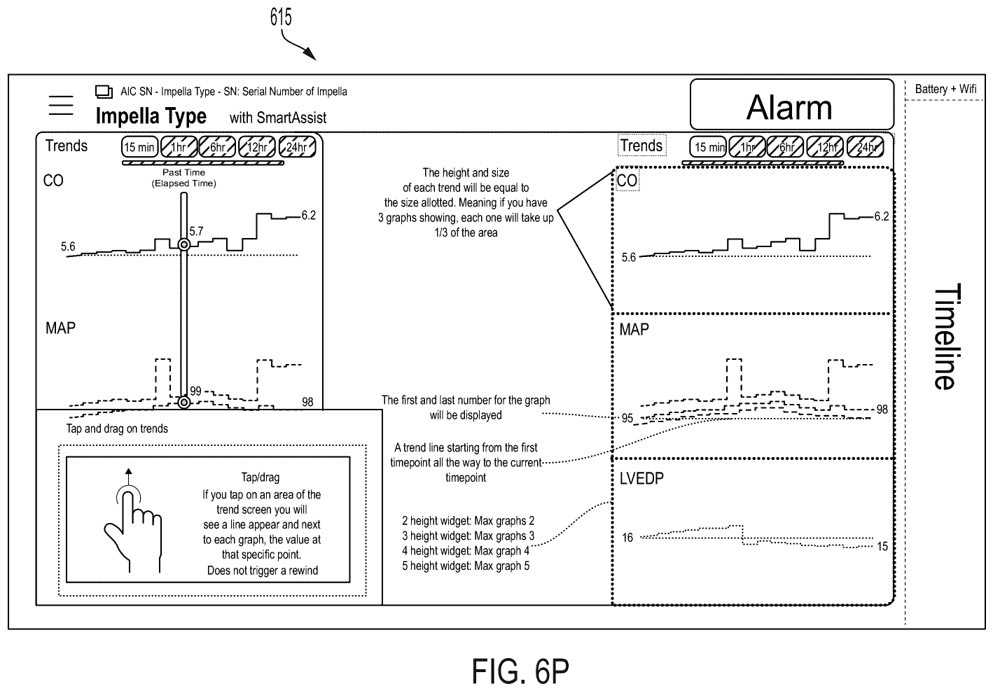

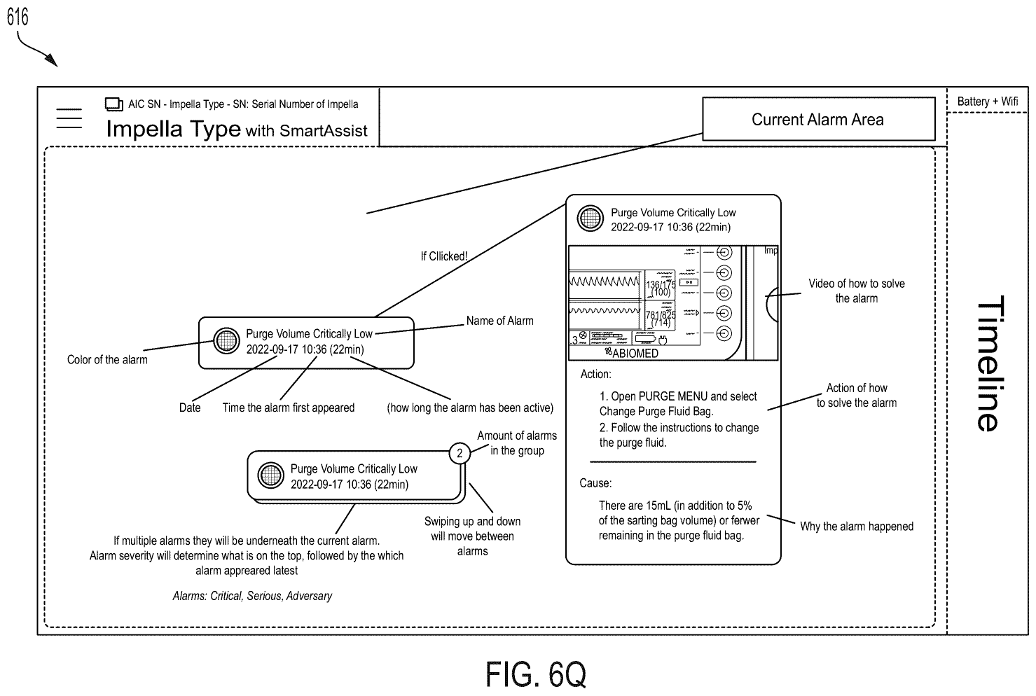

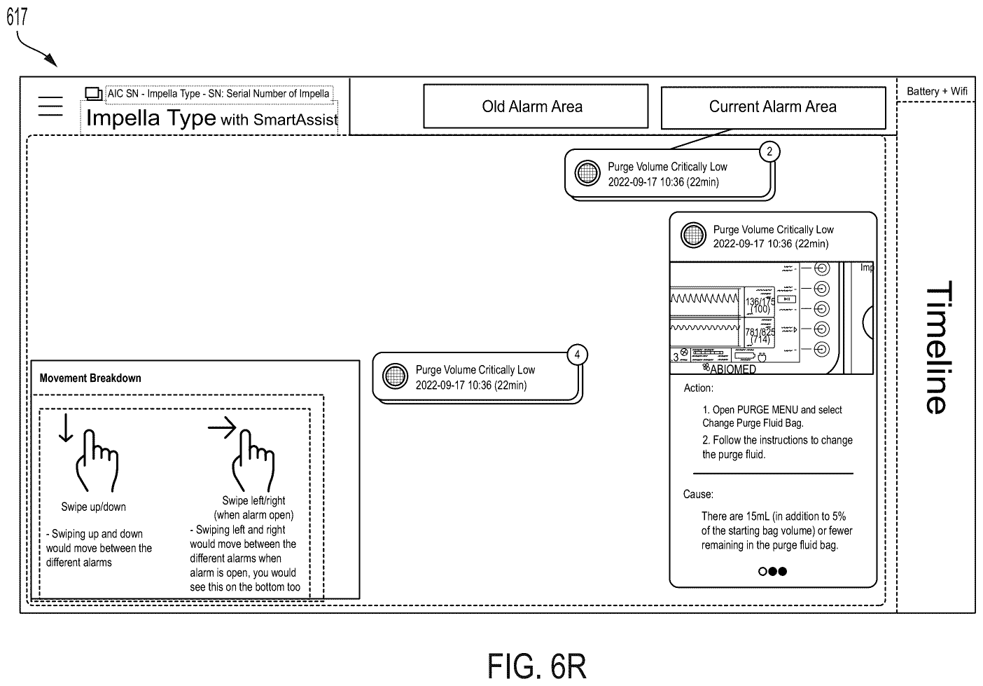

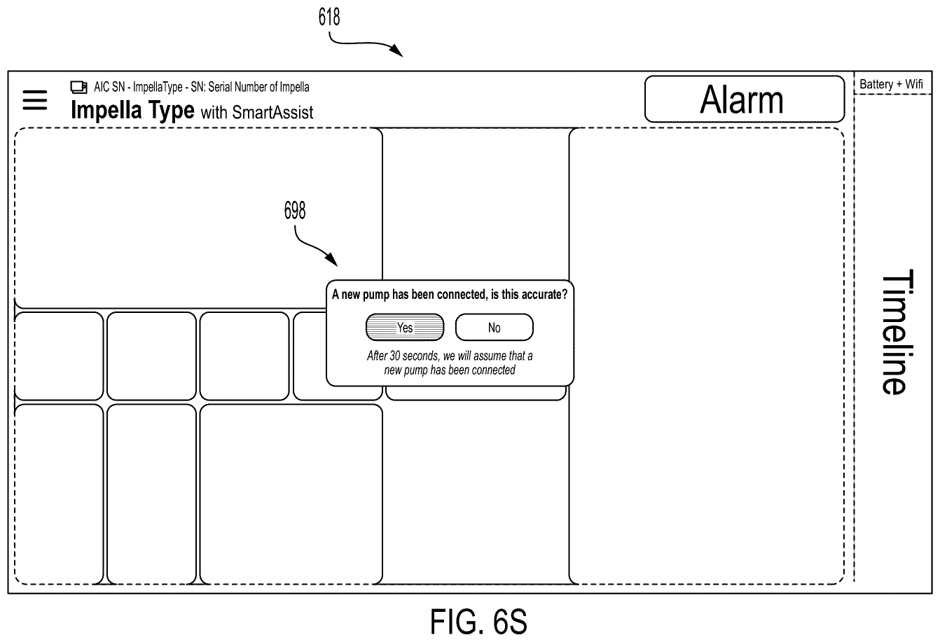

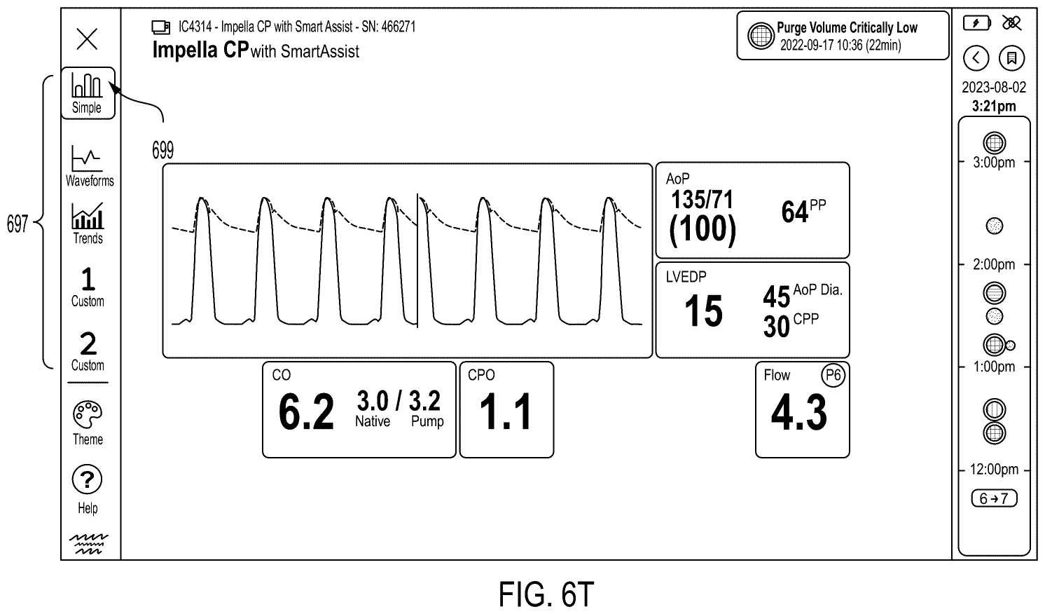

FIGS. 6A-6T illustrate exemplary portions of a user interface for auxiliary monitor 220 in accordance with some embodiments of the present disclosure. In the example user interfaces described in connection with FIGS. 6A-6T, information from a single connected patient monitoring device (e.g., a single connected AIC) is shown. However, it should be appreciated that any of the one or more techniques described above for presenting information from multiple connected patient monitoring devices may be used with any of the functionality described below as shown in FIGS. 6A-6T.

FIG. 6A illustrates a user interface 600 in which different portions of the user interface are configured to display different types of information associated with connected patient monitoring device, in accordance with some embodiments. As described in more detail below, some portions of the user interface may be configurable by a user such that the auxiliary monitor 220 may present a personalized view of the user interface specific to the preferences of the user, whereas other portions of the user interface may not be configurable by a user, but may instead present information about the connected patient monitoring device(s) and/or data received from and/or derived from the connected patient monitoring device(s). As shown in FIG. 6A, user interface 600 may include a title 634, which describes information for a connected patient monitoring device (which is an AIC in the example of FIG. 6A). User interface 600 may further include an alarm portion 632 within which different alarms received from the connected patient monitoring device and/or determined by the auxiliary monitor may be displayed.

Portions (e.g., displayed graphs, numerical values, alerts, etc.) of the user interfaces described herein may be updated as additional data is received by the auxiliary monitor 220 from one or more connected patient monitoring devices to provide a user of the auxiliary monitor with an up-to-date current view of patient monitoring information. The inventors have recognized and appreciated that it may be useful for a user of the auxiliary monitor 220 to be able to view historical information (i.e., from a time other than the current time) associated with data received and/or derived by the auxiliary monitor. Accordingly, in some embodiments, time series data received by the auxiliary monitor and/or derived by the auxiliary monitor may be stored by the auxiliary monitor. In some embodiments, the time series data may be transferred (e.g., via connectivity gateway 230) to a computing device for additional processing and/or to be stored in an electronic health record for the patient being monitored. User interface 600 may further include timeline portion 630 configured to enable the user to view information associated with the stored time series data. By storing time-series data and associated event/alarm data, as described herein, the user of the auxiliary monitor can review historical monitored patient information. Example functionality of timeline portion 630 is described further herein with reference to FIGS. 6B-6G.

In some embodiments, timeline portion 630, alarm portion 632, and title 634 may have fixed layouts on the user interface 600 that may not be configurable by a user. For instance, title 634 may always appear on the top of the user interface, alarm portion 632 may always appear to the right of the title 634, and the timeline portion 630 may always appear on the right side of the user interface 600. The remaining portion of user interface 600 may include a widget section 636 that has content and/or a layout that is configurable by a user of the auxiliary monitor. In the example shown in FIG. 6A, widget section 636 is shown having a particular layout that includes nine different widgets 638 of different sizes. In some embodiments, the layout of widget section 636 is customizable to enable a user to have the auxiliary display present information that is most useful for the user to monitor the status of the patient according to their preferences. For instance, in some embodiments, the user interface may be configured with one or more predefined layouts and/or one or more custom layouts that a user may configure for widget section 636. FIG. 6T illustrates an example of a user interface on which layout choices 697 are displayed along the left side of the user interface. As shown in FIG. 6T, some embodiments may provide a “simple” layout 699, an example which is shown in FIG. 6T, and/or other predefined layouts (e.g., a “waveform” layout, a “trend” layout” as shown in FIG. 6T). By configuring the user interface to have different layout choices, a user of the auxiliary monitor may quickly switch between different saved widget layout views, as desired.

In some embodiments, when designing custom layouts for widget section 636, a user may be able to select from different size widgets configured to display information for the same metric (e.g., CO, MAP, blood flow, etc.), albeit with different amounts and/or types of information displayed for the metric depending on the size of the selected widget. As described herein, the user interface for the auxiliary monitor may be configured, in some embodiments, as a touch interface that enables a user to rearrange and/or change widgets in widget section 636 according to their preferences or requirements at a particular point in time by dragging and dropping the widgets they prefer using touch.

As will be appreciated, although shown and described as being controlled via touch, in some embodiments, the auxiliary monitor may be configured to be voice activated and/or controlled. For example, the auxiliary monitor may be configured such that a user may talk to the auxiliary monitor during a procedure. In some embodiments, the auxiliary monitor may be configured to facilitate a support call. For example, in some embodiments, the clinician (either via talk or touch), can call for assistance or ask a question about a cardiac support device or additional device.

FIG. 6B illustrates a user interface 601 in which example additional details regarding aspects of timeline portion 630 is provided, in accordance with some embodiments. As shown in FIG. 6B, timeline portion 630 may include a timeline 640 of events with which a user of the auxiliary monitor may interact. For instance, timeline 640 may be scrollable such that when a user swipes up using a touch input, events from further in the past may be displayed in the timeline. In the example shown in FIG. 6B, the current time is shown on the top of the timeline 640 and past time is shown below the current time. It should be appreciated, however, that the timeline may be configured to show current vs. past time in any suitable manner. In some embodiments, as additional data is received from one or more connected patient monitoring devices and additional event data is generated, the events in timeline 640 may be automatically updated as time passes.

Timeline 640 may include a representation of events that were generated since a patient associated with timeline 640 was being monitored. The events represented in timeline 640 may include events generated by a connected patient monitoring device, events generated by the auxiliary monitor, and/or events generated in response to a user input. As shown in FIG. 6B, examples of events generated by a connected AIC may include P-level changes and alarms generated by the AIC. Alarms may be shown in the timeline 640 in different colors to represent a priority or importance of the alarm. In some embodiments, a user may interact with user interface 601 to add a bookmark at a particular point in timeline 640, and an event corresponding to the bookmark may be added to timeline 640. User-generated bookmarks may be associated with annotation information provided by the user to describe information about the point in timeline 640 with which the bookmark is associated.

FIG. 6C illustrates a user interface 602 that describes how timescales indicated on timeline 640 may change as time passes, in accordance with some embodiments. In some embodiments, a user may interact with timeline 640 using touch inputs (e.g., swipes, pinches, etc.) to move, expand, or contract a timescale of events displayed on timeline 640. Events displayed in timeline 640 may be associated with an event time and an event duration that is shown on timeline 640. For instance, an initial and total amount of time that a particular alarm was provided on a connected AIC may be represented on timeline 640. It should be appreciated that some events may not be associated with an event duration. For example, when a bookmark is generated in response to a user input, the bookmark may only be associated with a particular point in time on the timeline 640 and may not have an event duration.

FIG. 6D illustrates a user interface 603 that shows how timeline 640 may change in response to a user using different touch movements on the user interface to scroll the timeline back in time to observe past events, in accordance with some embodiments. For instance, as shown in FIG. 6D, the user may use touch movements to interact with timeline 640 to zoom in/out of a current time point, scroll timeline 640 backward and forward in time, and/or select a timepoint or event in the timeline 640. As the user interacts with timeline 640, the representation of the timeline may change to reflect the touch inputs provided by the user. In some embodiments, timeline 640 may be configured to automatically revert back to the view of the current time on the top of timeline 640 after a predetermined amount of time (e.g., 10 seconds, 20 seconds, 30 seconds, 1 minute, 5 minutes) has elapsed without the user interacting with the timeline 640.

FIG. 6E illustrates a user interface 604 that shows additional information about events in the timeline 640, in accordance with some embodiments. User interface 604 may be configured to represent timeline 640 in a normal view (e.g., as described above in connection with FIGS. 6B-6D) and an expanded view as shown in FIG. 6E. A user may toggle between the normal view and the expanded view by selecting a user interface element to expand or contract timeline 640. In the expanded view of timeline 640 shown in FIG. 6E, additional information for each of the events in the timeline may be displayed. For instance, an event type, a timepoint/time range, and/or an icon associated with each event may be shown in the expanded view of timeline 640. In response to a user selecting an event in the expanded view, the timeline 640 may go back to the timepoint associated with time associated with the selected event.

FIG. 6F illustrates a user interface 605 that shows an example of how timeline 640 may be displayed at the start of a case for a particular patient, in accordance with some embodiments. As shown in FIG. 6F, in some embodiments, a user may be able to view a filtered view of events in the timeline (e.g., by only displaying alarms or p-level changes) by interacting with a filter user interface element 642. As more events are added into the timeline 640, the user may be able to scroll to and/or select events in the past, as described herein.

As described herein, an auxiliary monitor may be configured to connect/disconnect from one or more patient monitoring devices in response to user input. The inventors have recognized and appreciated that it may be useful to capture such connection/disconnection events using timeline 640. FIG. 6G illustrates a user interface 606 that shows how connection/disconnection events may be represented in timeline 640, in accordance with some embodiments. In the example shown in FIG. 6G, a connected AIC device is disconnected at a certain point in time and then a new AIC (e.g., either the same AIC or a different AIC) is connected at a later point in time. By capturing events about connection and/or disconnection of different patient monitoring devices, a user of the auxiliary monitor may be better informed about the events that happened to the patient since the start of care for that patient.

As described in connection with FIG. 6A, a user interface for an auxiliary monitor configured according to the techniques described herein may include a widget section that is customizable by the user of the auxiliary monitor to provide a view of monitored data and/or calculated metrics aligned with the user's preferences. Each widget (also referred to herein as “tiles”) selected for display in the widget section may be associated with underlying functionality (e.g., one or more algorithms) used to compute the displayed graphics and numerical values on the user interface. As described herein, the widgets selected for display on the user interface of the auxiliary monitor may, for example, include widgets representing physiological parameters associated with the patient, widgets representing information about a timeline of monitoring of the patient, and widgets representing an operating state of one or more of the connected patient monitoring devices (e.g., an AIC).

FIG. 6H illustrates a user interface 607 that shows example widgets 650-656 for representing cardiac output (CO), in accordance with some embodiments. As shown in FIG. 6H, although each of widgets 650, 652, 654, and 656 shows information related to a patient's cardiac output, each of the widgets provides a different amount of information and/or represents the information differently. For example, widget 650 is a smaller widget that shows only the calculated cardiac output and no other information. Widgets 652 and 654 are slightly larger than widget 650 and show additional information about the cardiac output metric, specifically breaking down cardiac output into native output and output provided by a connected heart pump. Widget 656 provides even further detailed information about cardiac output by showing a time-based trend for cardiac output over a particular timescale (e.g., 2 minutes). Depending on how much detail the user would like to see on the auxiliary monitor for this particular metric and the available amount of available real estate within the widget section, the user can select one of the CO widgets for display on the user interface of the auxiliary monitor.

FIG. 6I illustrates a user interface 608 that shows example widgets 660-666 for representing left ventricular end diastolic pressure (LVEDP), in accordance with some embodiments. As shown in FIG. 6I, although each of widgets 660, 662, 664, and 666 shows information related to a patient's LVEDP value, each of the widgets provides a different amount of information and/or represents the information differently. For example, widget 660 is a smaller widget that shows only the calculated LVEDP value and no other information. Widgets 662 and 664 are slightly larger than widget 660 and show additional information about the LVEDP metric, specifically breaking down LVEDP into an aortic diastolic pressure (AoP Dia.) value and a coronary perfusion pressure (CPP) value measured by a connected heart pump. Widget 666 provides even further detailed information about the LVEDP metric by showing a time-based trend for LVEDP values over a particular timescale (e.g., 2 minutes). Depending on how much detail the user would like to see on the auxiliary monitor for this particular metric and the available amount of available real estate within the widget section, the user can select one of the LVEDP widgets for display on the user interface of the auxiliary monitor.

FIG. 6J illustrates a user interface 609 that shows example widgets 670-676 for representing mean arterial pressure (MAP), in accordance with some embodiments. As shown in FIG. 6J, although each of widgets 670, 672, 674, and 676 shows information related to a patient's MAP value, each of the widgets provides a different amount of information and/or represents the information differently. For example, widget 670 is a smaller widget that shows only the calculated MAP value and no other information. Widgets 672 and 674 are slightly larger than widget 670 and show additional information about the MAP metric, specifically a ratio of systolic to diastolic blood pressure and a pulse pressure value. Widget 676 provides even further detailed information about the MAP metric by showing a time-based trend for MAP values over a particular timescale (e.g., 2 minutes). Depending on how much detail the user would like to see on the auxiliary monitor for this particular metric and the available amount of available real estate within the widget section, the user can select one of the MAP widgets for display on the user interface of the auxiliary monitor.

FIG. 6K illustrates a user interface 610 that shows example widgets for displaying an elapsed time since a patient was being monitored using the auxiliary monitor, in accordance with some embodiments. As shown, different types of information may be displayed in the elapsed time widget depending on how much time has elapsed and a user's preferences.

FIG. 6L illustrates a user interface 611 that shows example widgets 680-686 for representing a flow rate through a heart pump associated with an AIC connected to the auxiliary monitor, in accordance with some embodiments. As shown in FIG. 6L, although each of widgets 680, 682, 684, and 686 shows information related to flow, each of the widgets provides a different amount of information and/or represents the information differently. For example, widget 680 is a smaller widget that shows only an average flow rate and a pump speed. Widgets 682 and 684 are slightly larger than widget 680 and show additional information about the flow including a minimum and maximum flow rate. Widget 686 provides even further detailed information about the flow by showing a time-based trend for flow rate over a particular timescale (e.g., 2 minutes). Depending on how much detail the user would like to see on the auxiliary monitor for this particular metric and the available amount of available real estate within the widget section, the user can select one of the flow rate widgets for display on the user interface of the auxiliary monitor.

FIG. 6M illustrates a user interface 612 that shows example widgets 690-696 for representing a cardiac power output (CPO) for a patient associated with a patient monitoring device (e.g., an AIC) connected to the auxiliary monitor, in accordance with some embodiments. As shown in FIG. 6M, although each of widgets 690, 692, 694, and 696 shows information related to CPO, each of the widgets provides a different amount of information and/or represents the information differently. For example, widget 690 is a smaller widget that shows only a CPO value. Widgets 692 and 694 are slightly larger than widget 690 and show additional information about the CPO value, breaking it down into a native CPO value and a pump CPO value. Widget 696 provides even further detailed information about the CPO metric by showing a time-based trend for CPO over a particular timescale (e.g., 2 minutes). Depending on how much detail the user would like to see on the auxiliary monitor for this particular metric and the available amount of available real estate within the widget section, the user can select one of the CPO widgets for display on the user interface of the auxiliary monitor.

As described in connection with FIGS. 3A-3J, a user interface for an auxiliary monitor may be configured to display trend information for one or more physiological metrics associated with a patient. In some embodiments, the trend information may be displayed as trend widgets in the widget section of the user interface. One of more of the trend widgets may be configurable to enable the user of the auxiliary monitor to view particular trend data of interest to the user.

FIG. 6N illustrates a user interface 613 that shows how a trend widget can be configured by a user of an auxiliary device, in accordance with some embodiments. As shown in FIG. 6N, the trend widget may enable a user to select different timescales for the trend (e.g., 15 min, 1 hr, 6 hr, 12 hr, 24 hr) and/or multiple metrics to display within the trend widget. By providing the user with a configurable interface for observing trends, the user can be provided with information that best enables them to monitor the patient to facilitate their care. In some embodiments, data represented in the trend widget is automatically updated as additional data is received by the auxiliary monitor.

FIG. 6O illustrates a user interface 614 that shows how a user can interact with the trend widget to zoom in and out of display of a trend metric, in accordance with some embodiments. As shown in FIG. 6O, a user may select a particular portion of the trend metric display and use touch inputs to expand or contract the trend metric display as desired. FIG. 6P illustrates a user interface 615 that shows how multiple trend graphs having different sizes can be displayed on the same trend widget, in accordance with some embodiments. As shown, a user may interact with the displayed trend information to compare values across metrics at the same timepoint. In some embodiments, multiple trend widgets can be selected and displayed in the widget section of the user interface.

As described herein, some connected patient monitoring devices are associated with a purge subsystem. FIG. 6Q illustrates a user interface 616 that shows alarm information for the purge subsystem of a connected patient monitoring device, in accordance with some embodiments. As shown in FIG. 6Q, when a particular alarm associated with the purge subsystem is selected, information about the alarm including when it first appeared and how long it has been active may be displayed. Additionally, if there are multiple associated alarms relating to the same event (e.g., low purge fluid), they may be indicated on the user interface 616 as shown in FIG. 6Q. In some embodiments, user interface 616 may be configured to provide the user of the auxiliary monitor with instructions on how to resolve the condition that caused the alarm. For example, in the example shown in FIG. 6Q, the user interface may be configured to show both the cause of the alarm relating to low purge fluid and also provide the user with instructions on how to resolve the alarm by changing the purge fluid.

FIG. 6R illustrates a user interface 617 that shows display of multiple alarms on the user interface, in accordance with some embodiments. As shown in FIG. 6R, when a user scrolls back in time (e.g., using the timeline), older alarms may be displayed to the left of current alarms, separated by a divider. A user may use touch inputs to scroll through alarms in the timeline, which may result in an update of the display provided on the user interface.

As described herein, the inventors have recognized and appreciated that one of the advantages of capturing a record (e.g., a longitudinal record) of patient monitoring using an auxiliary monitor is that an entire history of the patient's case can be recorded and later reviewed, if needed. In some instances, one or more components of a connected patient support device (e.g., a heart pump) may need to be disconnected and replaced with another patient support device. The inventors have recognized and appreciated that in such circumstances, it may be advantageous to associate metrics for operation of the new patient support device with metrics for operation of the old patient support device as a single instance of care. To this end, some embodiments of the present disclosure may be configured to detect when a new patient support device/patient monitoring device has been connected to the auxiliary monitor and the user may be prompted to confirm whether the newly-connected device is the same or different than the previously-connected device. FIG. 6S illustrates a user interface 618 that provides such a prompt 698. As will be appreciated, in embodiments in which the user selects “Yes” from this prompt, the auxiliary monitor may continue with the same configuration of widgets selected by the user, with the retrieved data corresponding to the newly-connected device being sent from the AIC to the auxiliary monitor.

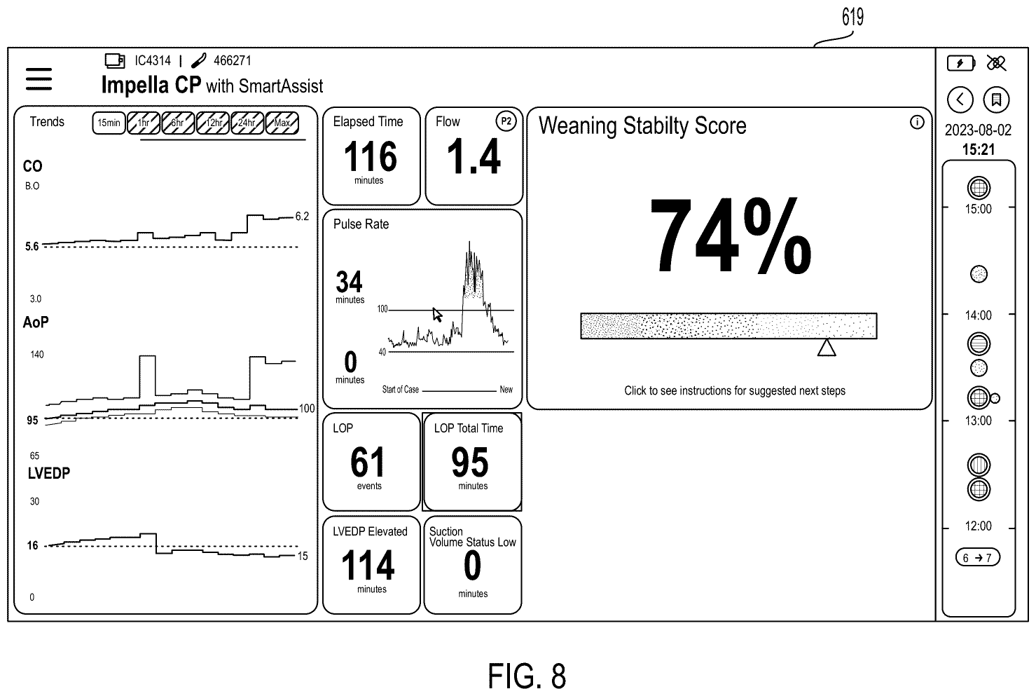

Still another user interface 619 can be seen in FIG. 8. As shown in this view, the user interface may be configured to illustrate a weaning stability score derived from one or more metrics. In some embodiments, metrics also may be displayed on the user interface, which may assist a user in understanding the weaning stability score. For example, in some embodiments, the user interface 619 may also show trends in cardiac output, aortic pressure, and left ventricular end diastolic pressure. The user interface also may show the time the patient has been using a support device (e.g., a pump), the flow, the pulse rate, the number of events in which the pump experienced a loss of pulsatility and the corresponding time, the time in which the left ventricular end diastolic pressure was above a prescribed value, and the time in which the pump may have experienced events of suction.

In some embodiments, the auxiliary monitor may be configured for data entry. For example, in some embodiments, a clinician may enter data into the auxiliary monitor for transfer to the patient's medical record (e.g., in the EHR). In other embodiments, the auxiliary monitor may be configured for data entry for a clinic trial. For example, instead of transferring data from the patient's medical record to an EHC, the auxiliary monitor may be configured to transfer data directly to the EHC. In such embodiments, the auxiliary monitor also may be configured to transfer data from the one or more cardiac support devices and/or the one or more other devices to the EHC as part of the study in addition to data being entered by the clinician. In still other embodiments, the auxiliary monitor may be configured to allow a clinician to “sign” the data for purposes of the study. In still other embodiments, the auxiliary monitor may be configured to ask specific questions of the clinician relative to the study that is being performed. In still other embodiments, the auxiliary monitor may be configured to administer the study protocol.

In some embodiments, the auxiliary monitor may be used to track procedures as they are happening, such as procedures happening in tandem with support via one or more of the cardiac support devices. For example, in an illustrative embodiment, the auxiliary monitor may be configured to track a percutaneous coronary intervention being performed by a clinician while the patient is receiving support from a cardiac support device. In such embodiments, the auxiliary monitor may be used for data entry during the procedure, as described herein. The auxiliary monitory also may be used to bookmark portions of the data feed on one or more of the tiles during certain portions of the procedure. The auxiliary monitor also may be able to track data received via one or more additional devices used as part of the procedure. In such embodiments, as will be appreciated, the auxiliary monitor may include one or more tiles configured to track such a procedure. For example, the clinician may press a Treatment Tile (e.g., a PCI tile) to note the start of the procedure and the end of the procedure. Such a Treatment Tile may be displayed with other tiles such that a clinician may view holistic data relating to the treatment and the one or more support or additional devices. For example, the clinician may view a risk score and/or a likelihood of recovery at the beginning and/or end of the procedure or during the procedure. The Treatment Tile also may be configured to show the severity of the procedure (e.g., easy or complex procedure) based on patient and/or device data. In some embodiments, information from the Treatment Tile may be considered in one or more algorithms configured to determine escalation and/or weaning of support.

As will be appreciated, the auxiliary monitor may be configured to transfer data relating to the Treatment Tile to the patient's heath record (e.g., EHR), as described herein.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. An auxiliary monitor for a patient monitoring system, the patient monitoring system including a first patient monitoring device and a second patient monitoring device, the auxiliary monitor comprising:
a communication interface configured to receive first data from the first patient monitoring device and second data from the second patient monitoring device; and
at least one computer processor programmed to:
display in a first portion of a user interface, an indication of the first data; and
display in a second portion of the user interface, an indication of the second data.

2. The auxiliary monitor of claim 1, wherein the at least one computer processor is further programmed to:
process the first data and the second data to generate third data; and
display in a third portion of the user interface, an indication of the third data.

3. The auxiliary monitor of claim 2, wherein the third data includes a likelihood of organ failure for a patient being monitored by the patient monitoring system.

4. (canceled)

5. The auxiliary monitor of claim 1, wherein the at least one computer processor is further programmed to:
display, in a third portion of the user interface, an alert, the alert determined based on the first data and/or the second data.

6.-7. (canceled)

8. The auxiliary monitor of claim 1, wherein
the first patient monitoring device comprises a first controller for a first cardiac support device including a first heart pump, and
the first data includes information describing operation of the first heart pump.

9. (canceled)

10. The auxiliary monitor of claim 8, wherein
the second patient monitoring device comprises a patient monitor, and
the second data includes vital signs measured by the patient monitor.

11. The auxiliary monitor of claim 8, wherein
the second patient monitoring device comprises a second controller for a second cardiac support device including a second heart pump, and
the second data includes information describing operation of the second heart pump.

12. (canceled)

13. The auxiliary monitor of claim 2, wherein the third data includes a likelihood of heart failure for a patient being monitored by the patient monitoring system.

14. (canceled)

15. The auxiliary monitor of claim 1, wherein
the first patient monitoring device is associated with a first patient, and
the second patient monitoring device is associated with a second patient.

16. The auxiliary monitor of claim 1, wherein the at least one computer processor is further programmed to:
issue a request via the communication interface to the first patient monitoring device and/or the second patient monitoring device, the request including a request for the first data and/or the second data,
wherein the communication interface is configured to receive the first data and/or the second data in response to the request.

17. The auxiliary monitor of claim 1, wherein the at least one computer processor is further programmed to:
issue a request via the communication interface to at least one network-connected processor, the request including a request to process the first data and/or the second data to generate processed data,
wherein the communication interface is configured to receive the processed data in response to the request, and
wherein the at least one computer processor is further programmed to display in a third portion of the user interface, an indication of the processed data.

18. The auxiliary monitor of claim 1, wherein the communication interface is configured to be connected to a connectivity gateway, the connectivity gateway communicatively coupling the communication interface to the first patient monitoring device and the second patient monitoring device.

19. The auxiliary monitor of claim 1, further comprising:
at least one storage device configured to store one or more of the first data, the second data, or data derived from the first data and/or the second data.

20. The auxiliary monitor of claim 17, wherein the processed data derived from the first data and/or the second data includes historical data associated with the first patient monitoring device and/or the second patient monitoring device.

21. A patient monitoring system, comprising:
a first patient monitoring device;
a second patient monitoring device;
an auxiliary monitor comprising at least one computer processor and a display; and
a connectivity gateway configured to communicatively couple the first patient monitoring device and the second patient monitoring device to the auxiliary monitor.

22. The patient monitoring system of claim 21, wherein the first patient monitoring device comprises a first controller for a first cardiac support device.

23. The patient monitoring system of claim 22, wherein the second patient monitoring device comprises a second controller for a second cardiac support device.

24. The patient monitoring system of claim 23, further comprising a third patient monitoring device communicatively coupled to the auxiliary monitor via the connectivity gateway,
wherein the third patient monitoring device comprises a patient monitor configured to monitor vital signs of a patient.

25. (canceled)

26. The patient monitoring system of claim 21, wherein the at least one computer processor of the auxiliary monitor is programmed to:
display in a first portion of a user interface presented on the display of the auxiliary monitor, an indication of first data received from the first patient monitoring device via the connectivity gateway;
display in a second portion of the user interface, an indication of second data received from the second patient monitoring device via the connectivity gateway;
process the first data and the second data to generate third data; and
display in a third portion of the user interface, an indication of the third data.

27-28. (canceled)

29. A method of monitoring a first cardiac support device and a second cardiac support device using an auxiliary monitor, the method comprising:
receiving, from the first cardiac support device, first data associated with operation of the first cardiac support device;
receiving, from the second cardiac support device, second data associated with operation of the second cardiac support device;
displaying, on a first portion of a user interface of the auxiliary monitor, an indication of the first data; and
displaying, on a second portion of the user interface, an indication of the second data.

30-45. (canceled)
